(19) United States
(12) Patent Application Publication
(10) Pub. No.: US 2021/0290672 A1
(43) Pub. Date: Sep. 23, 2021

(54) REGULATION OF TUMOR-ASSOCIATED T CELLS

(71) Applicant: UNIVERSITY HEALTH NETWORK, Toronto (CA)

(72) Inventors: Thuy-Linh NGUYEN, Toronto (CA); Pamela S. OHASHI, Toronto (CA); Sarah CROME, Toronto (CA)

(21) Appl. No.: 16/325,923

(22) PCT Filed: Aug. 17, 2017
(86) PCT No.: PCT/CA2017/000192

§ 371 Date: Feb. 15, 2019

Related U.S. Application Data
(60) Provisional application No. 62/375,970, filed on Aug. 17, 2016.

(51) Int. Cl. A61K 35/17; C07K 16/28; A61P 35/00

(52) U.S. Cl. CPC A61K 35/17; A61P 35/00; C07K 16/2803; C07K 16/2851

(57) ABSTRACT

There is described herein methods for improving tumor infiltrating lymphocyte (TIL) therapy by limiting the effect of innate lymphoid cells (ILCs) thereon. For example, in an aspect, there is provided a method of treating cancer in a patient in need thereof, comprising inhibiting the suppressive effect of CD56⁺CD3⁻ innate lymphoid cells (ILCs) on tumor infiltrating lymphocyte (TIL) propagation or expansion.

### FIELD OF THE INVENTION

The invention relates to tumor-infiltrating lymphocytes (TILs), compositions of these TILs between patients and how this relates to prognosis and methods for improving TIL expansion and function for the treatment of cancer.

### BACKGROUND OF THE INVENTION

Anti-tumor T cells are subject to multiple mechanisms of negative regulation¹⁻³. Recently, it was found that innate lymphoid cells (ILCs), including natural killer (NK) cells, regulate adaptive T cell responses⁴⁻⁶.

While once viewed as a homogeneous population whose function is to provide first-line defense against tumors and viruses, it is now appreciated that NK cells are part of a family of innate lymphocytes designated Innate Lymphoid Cells (ILCs) with diverse phenotypes and functions⁷. ILCs are currently classified into three groups⁷; Group 1 ILCs include both cytotoxic NK cells and ILC1s which produce IFN-γ but are not cytotoxic. Group 2 ILCs (ILC2) produce interleukins (IL)-4, IL-5, IL-9, IL-13, and Group 3 ILCs (ILC3) produce IL-22 alone or in combination with IL-17A. These definitions have been complicated by studies demonstrating that ILC3s cells can acquire an ILC1-like phenotype (ex-ILC3), that ILC1s exhibit cytotoxicity under certain conditions, and that markers previously used to differentiate ILC populations are often immune-context or tissue specific⁸. Therefore, properties that differentiate ILC populations are still poorly understood, particularly in humans.

A dynamic relationship between NK cells and other ILCs with T cells has been described^{4, 5}. Importantly, in addition to promoting T cell responses, NK cells can inhibit T cell-mediated immune responses in a variety of contexts, including autoimmunity⁹⁻¹² transplantation^{13, 14,} and viral infection¹⁵⁻²¹. The significance of NK cell-mediated regulation of T cells has recently been highlighted by mouse studies demonstrating that in vivo NK cell-depletion can improve anti-viral T cell responses and result in the clearance of lymphocytic choriomeningitis virus (LCMV) clone 13 that normally establishes a chronic infection^{19, 20}. In humans, NK cells from patients with chronic hepatitis B virus infections can kill HBV-specific CD8⁺ T cells in a TRAIL receptor-dependent manner²². In addition to direct cytotoxicity, NK cells may also have an impact on the adaptive immune response by altering cytokine production. Type 1 interferon treatment of hepatitis C virus-infected patients can lead to activation of NK cells and reduced production of IFN-γ by CD4⁺ T cells²³. Munneke et al. observed that the presence of activated ILCs corresponded with a reduced susceptibility to graft-versus-host disease²⁴, and ILC3s were shown to limit CD4⁺ T cell responses to intestinal commensal bacteria²⁵, supporting a role for ILCs in regulating adaptive responses.

### SUMMARY OF THE INVENTION

In an aspect, there is provided a method of treating cancer in a patient in need thereof, comprising inhibiting the suppressive effect of CD56⁺CD3⁻ innate lymphoid cells (ILCs) on tumor infiltrating lymphocyte (TIL) propagation or expansion.

In an aspect, there is provided a method of improving the anti-cancer effect of a population of cells comprising tumor infiltrating lymphocytes (TILs) comprising depleting CD56⁺CD3⁻ innate lymphoid cells (ILCs) from said population.

In an aspect, there is provided a method of improving the anti-cancer effect of a population of cells comprising tumor infiltrating lymphocytes (TILs) comprising adding to said population a compound that decreases the suppressive effect of CD56⁺CD3⁻ innate lymphoid cells (ILCs) on tumor infiltrating lymphocyte (TIL) propagation, expansion or function.

In an aspect, there is provided a method of treating cancer in a patient in need thereof, comprising administering to the patient a therapeutically effective amount of a compound that decreases the suppressive effect of CD56⁺CD3⁻ innate lymphoid cells (ILCs) on tumor infiltrating lymphocyte (TIL) propagation, expansion or function.

In an aspect, there is provided a method of treating cancer in a patient in need thereof, comprising administering to the patient a therapeutically effective amount of antibodies against NKG2D, NKp30 or NKp46.

In an aspect, there is provided antibodies against NKG2D, NKp30 or NKp46 for use in the treatment of cancer.

In an aspect, there is provided a use of antibodies against NKG2D, NKp30 or NKp46 in the preparation of a medicament for the treatment of cancer.

In an aspect, there is provided a pharmaceutical composition comprising of antibodies against NKG2D, NKp30 or NKp46 and a pharmaceutically acceptable carrier.

In an aspect, there is provided a method of predicting a patient outcome in a patient having cancer, or patient being treated or having been treated for cancer, preferably time to recurrence or overall survival, comprising measuring the presence of CD56⁺CD3⁻ innate lymphoid cells (ILCs); and predicting a patient outcome, wherein a relatively higher presence of ILCs is associated with a worse patient outcome and a relatively lower presence of ILCs is associated with a better patient outcome.

### BRIEF DESCRIPTION OF FIGURES

These and other features of the preferred embodiments of the invention will become more apparent in the following detailed description in which reference is made to the appended drawings wherein:

FIG. 1. ILCs can suppress the expansion of tumor-infiltrating lymphocytes. (a) Multiple TIL cultures from individual high-grade serous cancer (HGSC) specimens were expanded. TIL growth and proportions of CD3⁻CD56⁺ cells were determined. “Fast” expansion rates refer to TIL cultures that yielded >30×10⁶ cells on or before 4 weeks, “slow” refers to TIL cultures which achieved 2-29×10⁶ cells by 4 weeks, and “no” refers to cultures which had cell yields below 2×10⁶ cells. (b-e) Percentage of cells positive for indicated lineage markers in fast or slow/no expansion cultures were analyzed. The percent of cells in TIL cultures are shown for (b) CD56⁺CD3⁻ cells and CD3⁺CD56⁻ cells (Fast n=51, Slow/No n=49), (c) CD3⁻CD56⁺ cells (Fast n=51, Slow/No n=49), (d) CD14⁺ cells (Fast n=40, Slow/No n=29), and CD19⁺ cells (Fast n=40 , Slow/No n=37), and (e) CD4⁺ T cells and CD8⁺ T cells (Fast n=37, Slow/No n=36). For c-e, each circle represents an independent TIL culture. (f-g) TIL from slow/no expansion cultures were stimulated with anti-CD3, feeder cells and IL-2, with and without depletion of CD3⁻CD56⁺ cells. The expansion yields were calculated by combining cell counts with flow cytometric analysis of the types of cells present following stimulation. Each circle represents a different patient evaluated (n=7). (h) Flow cytometry-sorted CD8⁺ and CD4⁺ TIL from slow/no expansion TIL cultures were labeled with cell proliferation dye and activated with anti-CD3 and anti-CD28. Expansion in the presence or absence of sorted autologous CD3⁻CD56⁺ cells from slow/no expansion TIL cultures was assessed at 72 hours. Each circle represents a different patient evaluated (n=8). Significance as determined by Mann Whitney test for c-e, and Wilcoxon matched-pairs signed rank test for f-h, is indicated or if not significant, denoted by n.s.

FIG. 2. T cell cytokine production is altered in cultures containing regulatory ILCs (a) Cytokine production by TIL cultures that had slow/no expansion and high proportions of CD56⁺CD3⁻ or fast-expanding TIL with low proportions of CD3⁻CD56⁺ cells was assessed by cytometric bead assay (n=16). Each circle represents an individual TIL culture from a different patient. (b-d) Flow cytometry-sorted CD8⁺ and CD4⁺ TIL from slow/no expansion cultures were labeled with cell trace and activated with anti-CD3 and anti-CD28 in the presence or absence of sorted autologous CD56⁺CD3⁻ cells. Intracellular cytokine production was assessed at 72 hr. (b) Representative and (c-d) average IFN-α and TNF-α production in CD4⁺ and CD8⁺ TIL in the presence or absence of CD56⁺CD3⁻ cells (n=7). Statistical significance was determined by Mann Whitney test for a or Wilcoxon matched-pairs signed rank test for c-d is indicated, or if not significant is denoted by n.s.

FIG. 3. Regulatory ILCs have unique properties. RNA-seq was performed on flow cytometry-sorted CD56⁺CD3⁻ cells in slow/no expansion TIL cultures that suppressed TILs (regulatory CD56⁺CD3⁻) or CD56⁺CD3⁻ cells from fast expansion TIL cultures that did not suppress TILs (CD56⁺CD3⁻). (a) Heat map representation of statistically significant differences in gene expression between CD56⁺CD3⁻ cells and regulatory CD56⁺CD3⁻ cells. Color scale represents the per gene Z-score, number of standard deviations away from mean gene expression across all samples. Genes selected based on multiple testing adjusted p-value<0.05 and log2 fold change>1. Heat map representation of expression of (b) NK cell and ILC-related molecules, (c) KIRs, and, (d) transcription factors by regulatory CD56⁺CD3⁻ cells and CD56⁺CD3⁻ cells. Color scale represents log-transformed, upper quartile normalized transcript abundance measured in transcripts per million (TPM). (e) Flow cytometry-sorted regulatory CD56⁺CD3⁻ cells were stimulated with IL-2, and supernatants collected after 24 hours. Cytokine expression was measured by cytometric bead assay (n=6 patients). Averages presented as mean±s.e.m. (f-i) Intracellular cytokine production on flow cytometry-sorted regulatory CD56⁺CD3⁻ cells and CD56⁺CD3⁻ cells were assessed after a 16 hour stimulation with IL-2 and re-stimulation with PMA and ionomycin. (f) Representative, and (g) average, production of TNF-α (f) Repre by regulatory CD56⁺CD3⁻ cells (n=4 patients) and CD56⁺CD3⁻ cells (n=4 patients)□ (h) Representative expression of IL-22, IL-9 and IL-17A and (i) average production of IL-22, by regulatory CD56⁺CD3⁻ cells (n=5 patients) and CD56⁺CD3⁻ cells (n=4 patients). Statistical significance in g and i was determined by Mann Whitney test.

FIG. 4. Regulatory ILCs limit T cell expansion via NCRs and their presence is associated with recurrence free survival. (a) Heat map representation of expression of granzymes and perforin on flow cytometry-sorted CD56⁺CD3⁻ cells from slow/no expansion TIL cultures that suppressed TILs (regulatory CD56⁺CD3⁻) or CD56⁺CD3⁻ cells from fast expansion TIL cultures that did not suppress TILs (CD56⁺CD3⁻). Color scale represents log-transformed, upper quartile normalized transcript abundance measured in transcripts per million (TPM). (b) Regulatory CD56⁺CD3⁻ cells and peripheral blood NK cells from healthy donors (PB NK cell) were isolated by flow cytometry-based sorting and co-cultured with K562 cells in the presence of IL-2. CD107a expression by CD56⁺CD3⁻ cells and cell death of K562 cells were analyzed after 6 hours. (b) Representative and (c) average CD107a expression by regulatory CD56⁺CD3⁻ cells (n32 5 patients) or PB NK cells (n=4 healthy donors). (d) Average percentage of K562 cells positive for viability dye represented as fold increase in cell death when co-cultured with regulatory CD56⁺CD3⁻ cells (n=5 patients) or PB NK cells (n=4 healthy donors). (e) TIL expansion and cytokine production was analyzed in the presence of supernatants obtained from culturing flow-cytometry sorted regulatory CD56⁺CD3⁻ cells. (e) Percentage suppression of CD4⁺ and CD8⁺ TIL. Each circle represents a TIL culture from a different patient (n=5). (f) Representative intracellular IFN-α and TNF-α production in CD4⁺ and CD8⁺ TIL expanded in the presence or absence of supernatants from regulatory CD56⁺CD3⁻ cells for 5 days (n=4). (g-h) Representative expression and mean fluorescence intensity (MFI) of NKG2D, NKp30, and NKp46 expression by regulatory CD56⁺CD3⁻ cells and CD56⁴CD3⁻ cells from independent TIL cultures (n=16) (i) Expansion yields of TIL in the presence or absence of anti-NKG2D, anti-NKp30, or anti-NKp46 antibodies were compared following stimulation with feeder cells, anti-CD3, and IL-2. Each circle represents expansion cultures from a different patient (n=7). (j) Recurrence-Free Survival (RFS) was analyzed in HGSC patients whose TIL cultures contained regulatory CD56⁺CD3⁻ cells (n=6) or did not contain regulatory CD56⁺CD3⁻ cells (n=10). Patients were chemotherapy-naïve at the time of TIL isolation and surgery achieved optimal debulking. Statistical significance was determined by Mann Whitney test for c-d and g-h, Wilcoxon matched-pairs signed rank test for e and i, and Log-rank (Mantel-Cox) test in j. Statistical significance is indicated, or if not significant indicated by n.s. FIG. 5: Regulatory Innate Lymphoid cells express various checkpoint molecules.

FIG. 5. Analysis of CD56⁺CD3⁻ cells in fast or slow/no expansion TIL cultures. Flow cytometry gating strategy for analysis of proportions of CD56⁺CD3⁻ cells and T cells in the TIL characterization was performed as indicated. TIL which were CD19⁻ CD14⁻ and negative for fixable viability dye were analyzed for proportions of CD56 and CD3. CD3⁺ T cells were also examined for proportions of CD4⁺ and CD8⁺ T cells (See FIG. 1). Following functional characterization including cell growth and suppressive capacity, cryopreserved samples from TIL cultures were thawed and a secondary analysis was performed to characterize the transcriptome and/or the phenotype of CD56⁺CD3⁻ cells. Refer to FIG. 3, FIG. 4 and Supplementary FIG. 4-9 for this characterization of CD56⁺CD3⁻ cells.

FIG. 6. Representative suppression of TIL expansion. Flow cytometry-sorted CD3⁺ TIL from slow/no expansion TIL were labeled with cell trace and activated with anti-CD3 and anti-CD28 for 72 hrs. The number of labeled CD3⁺ TIL that were CD4⁺ or CD8⁺ TIL was determined in the presence or absence of sorted autologous CD56⁺CD3⁻ cells and percent suppression calculated as indicated. Average suppression by CD56⁺CD3⁻ cells from slow/no expansion TIL cultures displayed in FIG. 1f.

FIG. 7. CD56⁺CD3⁻ cells from fast-expanding TIL cultures or from PB do not suppress expansion of autologous T cells. (a) Expansion yields of fast-expanding TIL with and without depletion of CD3⁻CD56⁺ cells was calculated by combining cell counts with flow cytometric analysis of the types of cells present following stimulation with feeder cells, anti-CD3 and IL-2 (n=4). (b) Flow cytometry-sorted CD3⁺ peripheral blood (PB) T cells were labeled with cell trace and activated with anti-CD3 and anti-CD28. Expansion in the presence or absence of sorted autologous PB NK cells was assessed at 72 hours (n=3). Statistical significance determined by Wilcoxon matched-pairs signed rank test. P-value of n.s. indicates not significant.

FIG. 8: Summaries of RNA-Seq data quality control metrics. Transcriptome analysis of flow cytometry sorted CD56⁺CD3⁻ cells in slow/no expansion TIL cultures that suppressed TILs (regulatory CD56⁺CD3⁻) or CD56⁺CD3⁻ cells from fast expansion TIL cultures that did not suppress TILs (CD56⁺CD3⁻) were examined by RNA-Seq. Metrics collected with RNA-seQC of RNA. (a) Estimated Library Size, based on number of expected unique RNA fragments assuming Poisson distribution given the total read number and duplication rate in the sample. (b) Percentage of reads duplicated within the total number of reads sequenced. (c) Percentage of ribosomal RNA reads to the total number of reads sequenced. (d) Average fragment length sequenced. (e) Percentage of mapped reads that mapped to exonic, intronic and intergenic regions of the reference genome. (f) Per base transcript coverage of low-expressed transcripts. (g) Per base transcript coverage of medium-expressed transcripts. (h) Per base transcript coverage of high-expressed transcripts

FIG. 9. Phenotype of regulatory CD56⁺CD3⁻ cells from TIL cultures. CD56⁺CD3⁻ CD14⁻CD19⁻ cells from slow/no expansion TIL cultures (regulatory CD56⁺CD3⁻) or fast-growing TIL cultures (CD56⁺CD3⁻) were analyzed by flow cytometry for expression of NK cell and ILC-associated molecules as indicated. Representative and average expression of (a) CD16 (b) CD7, and (c) CD57. (d) Average percentage positive of CD94, CD94/NKG2C and CD94/NKG2A. (e) Representative and average mean fluorescence intensity (MFI) of NKp44 expression (f) Average expression of indicated killer-cell immunoglobulin-like receptors (KIRs). Each circle represents an individual TIL culture. Statistical significance as determined by Mann Whitney test indicated, or if not significant, denoted by n.s.

FIG. 10. Ex vivo expression of NK cell and ILC-associated molecules on CD56⁺CD3⁻cells from TIL prior to expansion. Ex vivo CD56⁺CD3⁻CD14⁻CD19⁻ cells from HGSC patients (ex vivo CD56⁺CD3⁻) and peripheral blood NK cells (PBMC NK) were analyzed by flow cytometry for expression of NK cell and ILC-associated molecules. The phenotype of these cells following expansion was also monitored (see

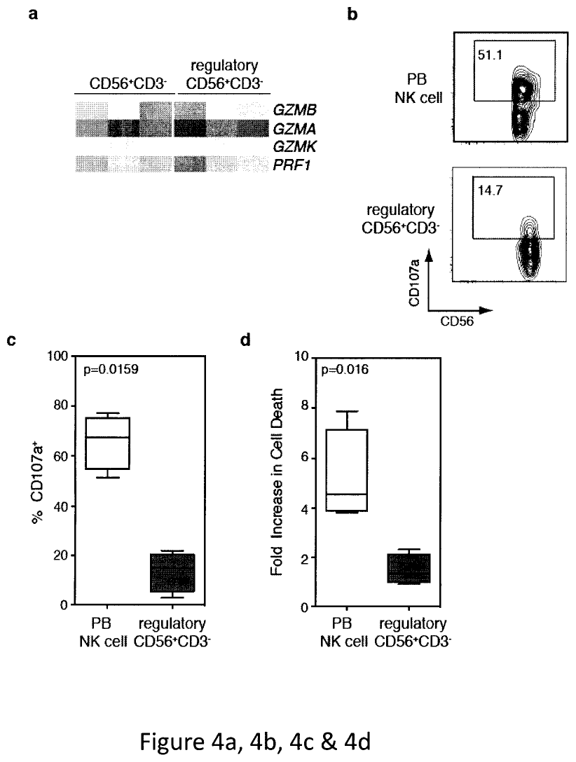

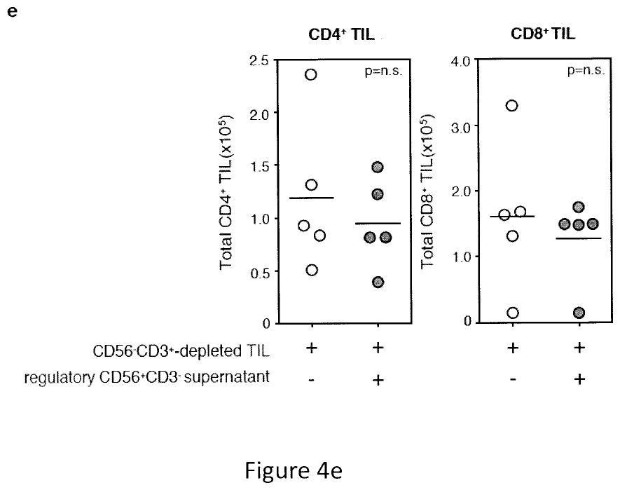

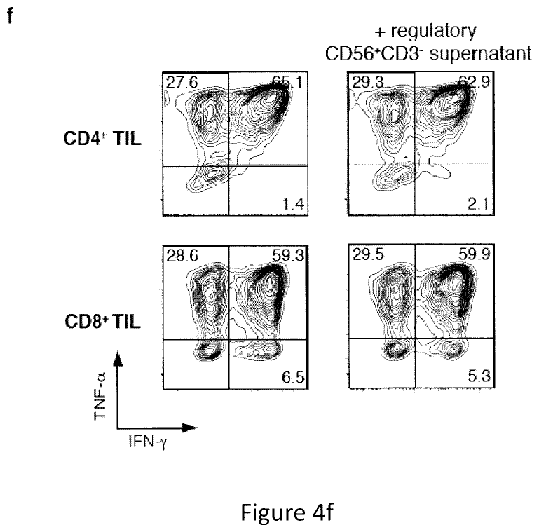

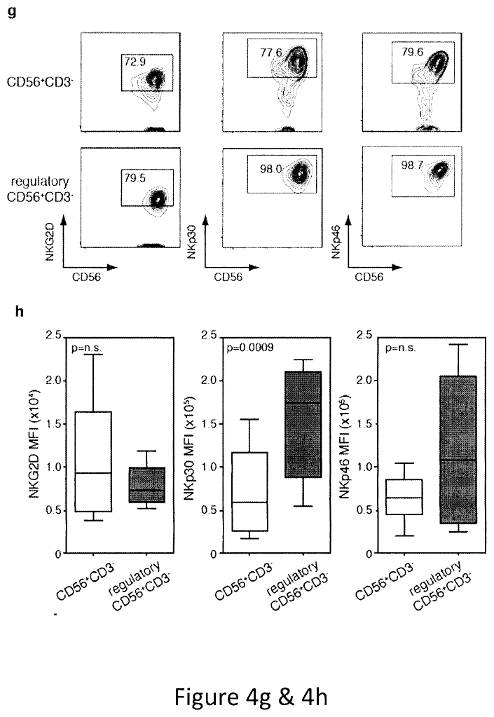

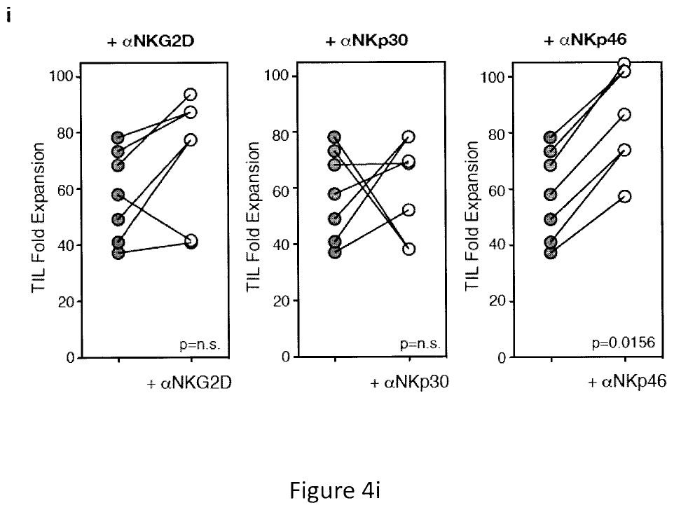

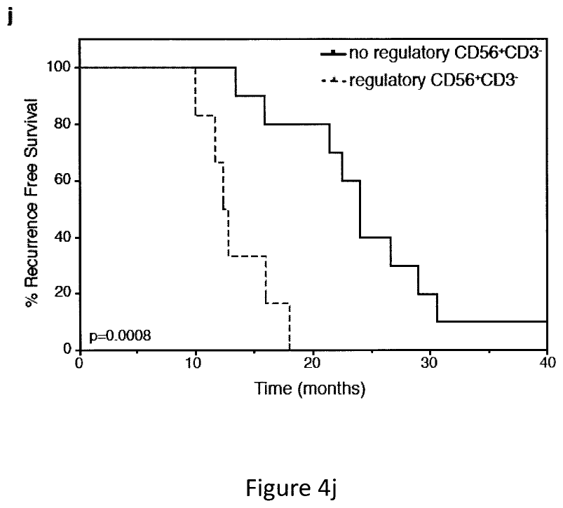

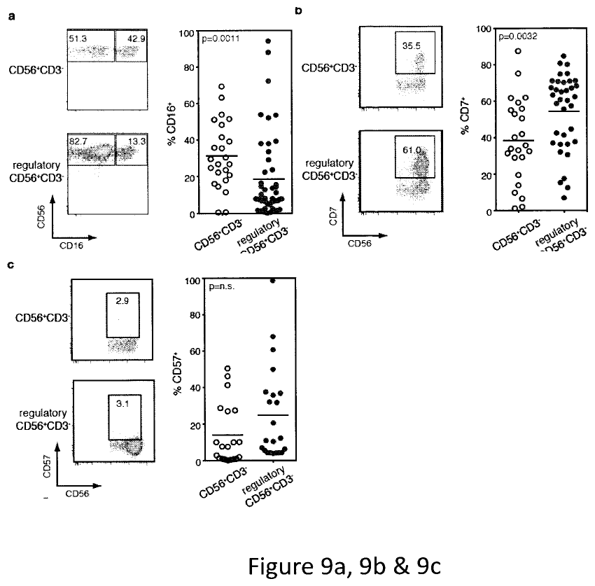

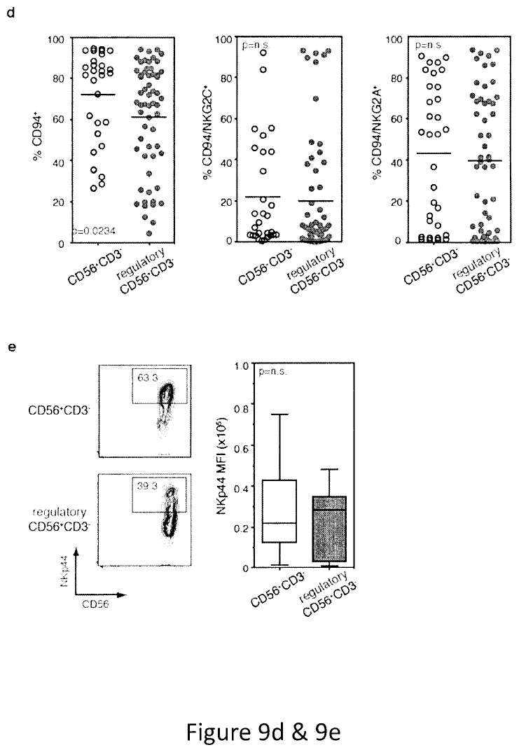

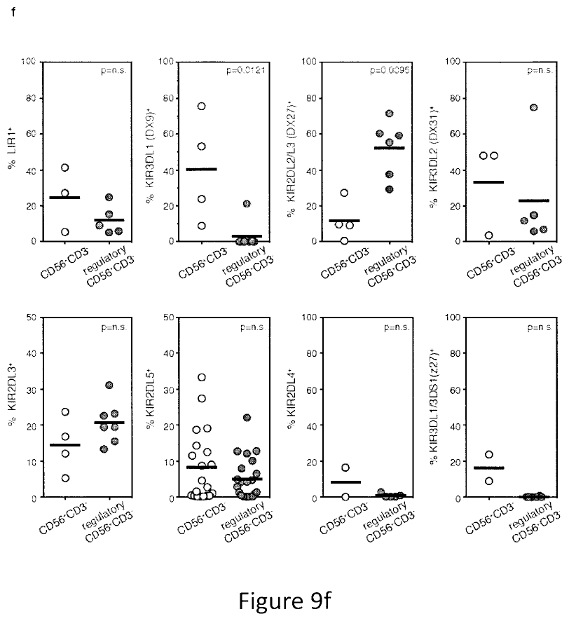

FIG. 4g-i and FIG. 9). Percentage positive for expression of (a) CD16 (n=11), and (b) CD57 (n=5-12). (c) Percentage positive for expression of NKp30, NKp44 and NKp46 (n=4-7). (d) Percentage positive for expression of CD94 family (n=4-7). Statistical significance as determined by Mann Whitney test indicated, or if not significant, denoted by n.s.

FIG. 11. Regulatory CD56⁺CD3⁻ cells do not express FOXP3. CD56⁺CD3⁻CD14⁻ CD19⁻ cells from slow/no expansion TIL cultures (regulatory CD56⁺CD3⁻) or CD56⁺CD3⁻CD14⁻CD19⁻ cells from fast-expanding TIL cultures (CD56⁺CD3⁻), were analyzed by flow cytometry for expression of FOXP3 and CD25. (a) Representative FOXP3 expression by regulatory CD56⁺CD3⁻ cells and CD56⁺CD3⁻ cells (n=8). (b) Representative FOXP3 staining in peripheral blood CD3⁺ T cells.

FIG. 12. Regulatory CD56⁺CD3⁻ cells secrete CCL3. CD56⁺CD3⁻CD19⁻CD14⁻ cells from slow/no-expansion TIL cultures (regulatory CD56⁺CD3⁻ cells) were sorted by flow cytometry, stimulated with IL-2, and supernatants collected after 24 hours. Chemokine expression was measured by cytometric bead assay.

FIG. 13. Regulatory CD56⁺CD3⁻ cells do not have increased transcript level expression of IL10 or TGFB1. CD3⁻CD56⁺CD19⁻CD14⁻ cells from slow/no-expansion TIL cultures (regulatory CD56⁺CD3⁻) and CD3⁻CD56⁺CD19⁻CD14⁻ cells from fast expansion TIL cultures (CD56⁺CD3⁻) were sorted by flow cytometry and transcriptome analysis was performed. Log-transformed, upper quartile normalized transcript abundance measured in transcripts per million (TPM). Statistical significance determined using the generalized linear model in DESeq2. P-value of n.s. indicates not significant.

FIG. 14. Correlation between CD56 expression and patient outcome in HGSC patients. CD56 expression in the Tothill dataset of 215 high-grade serous tumors was ranked from high to low. (a) Recurrence-free survival data, and, (b) overall survival data, was analyzed in the top 50% CD56-expressing tumors (CD56 high, n=107) and the bottom 50% CD56-expressing tumors (CD56 low, n=107). Median RFS was 14 months in the top CD56-expressing tumors compared to 18 months in patients with low CD56 expression (p=0.0155). There was also a 10-month decrease in median overall survival (48 months versus 38 months) in patients expressing high CD56 levels, which was significant (p=0.0223) using the Gehan-Breslow-Wilcoxon test.

FIG. 15. Correlation between CD56⁺CD3⁻ cells in TIL culture and poor expansion yields of melanoma and breast TIL. Multiple TIL cultures from individual melanoma or breast cancer specimens were expanded. TIL growth and proportions of CD3⁻CD56⁺ cells were determined. “Fast” expansion rates refer to TIL cultures that yielded >30×10⁶ cells on or before 4 weeks, “slow” refers to TIL cultures which did not expand or only achieved 2-29×10⁶ cells by 4 weeks, (a) Percentages of CD3⁻CD56⁺ cells in melanoma TIL cultures with fast or slow expansion. (b) Percentages of CD3⁻CD56⁺ cells in breast cancer TIL cultures with fast or slow expansion.

### DETAILED DESCRIPTION

In the following description, numerous specific details are set forth to provide a thorough understanding of the invention. However, it is understood that the invention may be practiced without these specific details.

We identified a novel ILC population that regulates tumor-infiltrating lymphocytes (TIL) from high-grade serous ovarian tumors, defined their suppressive capacity in vitro, and performed a comprehensive analysis of their phenotype. Notably, the presence of this regulatory CD56⁺CD3⁻ population (hereafter referred to as regulatory ILC) in TIL cultures correlated with reduced T cell numbers, and further functional studies demonstrated that these cells suppress TIL expansion and alter their cytokine production. Transcriptome analysis and phenotypic characterization determined that this regulatory ILC population has a distinct phenotype from previously identified ILCs. Regulatory ILCs exhibited low cytotoxic activity and produced interleukin (IL)-22, yet expressed many receptors associated with conventional NK cells. NKp46 was highly expressed by these cells, and addition of anti-NKp46 antibodies to TIL cultures abrogated the ability of regulatory ILCs to suppress T cell expansion. Importantly, the presence of regulatory ILCs in TIL cultures corresponded with a striking reduction in the time to disease recurrence in patients. These studies demonstrate that a previously uncharacterized ILC population regulates tumor-associated T cells.

In a further aspect, there is provided a method of improving the anti-cancer effect of a population of cells comprising tumor infiltrating lymphocytes (TILs) comprising adding to said population a compound that decreases the suppressive effect of CD56⁺CD3⁻ innate lymphoid cells (ILCs) on tumor infiltrating lymphocyte (TIL) propagation, expansion or function.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

As used herein, “Innate lymphoid cells” or “ILCs” refer to the group of innate immune cells that belong to the lymphoid lineage (lymphocytes) but do not respond in an antigen-specific manner, as they lack a B or T cell receptor. As noted above, ILCs are currently classified into three groups; Group 1 ILCs include both cytotoxic NK cells and ILC1s which produce IFN-γ but are not cytotoxic. Group 2 ILCs (ILC2) produce interleukins (IL)-4, IL-5, IL-9, IL-13, and Group 3 ILCs (ILC3) produce IL-22 alone or in combination with IL-17A.

As used herein, “tumor-infiltrating lymphocytes” or “tumour infiltrating lymphocytes” (TILs), are white blood cells that have left the bloodstream and migrated into a tumor. They are mononuclear immune cells, a mix of different types of cells (i.e., T cells, B cells, NK cells, etc) in variable proportions, T cells typically being the most abundant cells. Therapeutic use of TILs is commonly described as use of T cells found in a tumor mass to treat cancer. They can often be found in the stroma and within the tumour itself. TILs are implicated in killing tumor cells and the presence of lymphocytes in tumors is often associated with better clinical outcomes.

The present methods would be useful in therapies for any cancer that are treatable or can be targeted with TILs, and may include, without limitation, adrenal cancer, anal cancer, bile duct cancer, bladder cancer, bone cancer, brain/cns cancer, brain/cns cancer, breast cancer, cervical cancer, colon/rectum cancer, endometrial cancer, esophagus cancer, eye cancer, gallbladder cancer, gastrointestinal cancer, kidney cancer, laryngeal and hypopharyngeal cancer, liver cancer, lung cancer, malignant mesothelioma, nasal cavity and paranasal sinus cancer, nasopharyngeal cancer, neuroblastoma, oral cavity and oropharyngeal cancer, ovarian cancer, pancreatic cancer, penile cancer, pituitary tumors, prostate cancer, retinoblastoma, salivary gland cancer, sarcoma, skin cancer, small intestine cancer, stomach cancer, testicular cancer, thymus cancer, thyroid cancer, uterine sarcoma, vaginal cancer, vulvar cancer, or wilms tumor.

In some embodiments, the cancer is a cancer associated with poor TIL expansion.

In some embodiments, the cancer is melanoma, breast cancer, prostate cancer, or ovarian cancer, preferably serous (high grade) ovarian cancer.

In some embodiments, the ILCs are at least one of CD56^{hi}, CD16⁻, IL-22⁺, CD94⁺, NKG2D⁺, KIR⁺, NKp44⁻ ex vivo, NKp30⁺, NKp46⁺, preferably all of the foregoing

In some embodiments, the compound is an antibody against a surface marker on CD56⁺CD3⁻ ILCs, preferably NKG2D, NKp30, NKp46, or combinations thereof.

The terms “antibody” and “immunoglobulin”, as used herein, refer broadly to any immunological binding agent or molecule that comprises a human antigen binding domain, including polyclonal and monoclonal antibodies. Depending on the type of constant domain in the heavy chains, whole antibodies are assigned to one of five major classes: IgA, IgD, IgE, IgG, and IgM. Several of these are further divided into subclasses or isotypes, such as IgG1, IgG2, IgG3, IgG4, and the like. The heavy-chain constant domains that correspond to the difference classes of immunoglobulins are termed α, δ, ε, γ and μ, respectively. The subunit structures and three-dimensional configurations of different classes of immunoglobulins are well known.

Generally, where whole antibodies rather than antigen binding regions are used in the invention, IgG and/or IgM are preferred because they are the most common antibodies in the physiological situation and because they are most easily made in a laboratory setting.

The “light chains” of mammalian antibodies are assigned to one of two clearly distinct types: kappa (κ) and lambda (λ), based on the amino acid sequences of their constant domains and some amino acids in the framework regions of their variable domains.

There is essentially no preference to the use of κ or λ light chain constant regions in the antibodies of the present invention.

As will be understood by those in the art, the immunological binding reagents encompassed by the term “antibody” extend to all human antibodies and antigen binding fragments thereof, including whole antibodies, dimeric, trimeric and multimeric antibodies; bispecific antibodies; chimeric antibodies; recombinant and engineered antibodies, and fragments thereof.

The techniques for preparing and using various antibody-based constructs and fragments are well known in the art. Diabodies, in particular, are further described in EP 404, 097 and WO 93/11161.

Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, T and Abs, ds-scFv, dimers, minibodies, diabodies, bispecific antibody fragments and other fragments can also be synthesized by recombinant techniques or can be chemically synthesized. Techniques for producing antibody fragments are well known and described in the art.

The human antibodies or antibody fragments can be produced naturally or can be wholly or partially synthetically produced. Thus the antibody may be from any appropriate source, for example recombinant sources and/or produced in transgenic animals or transgenic plants, or in eggs using the IgY technology. Thus, the antibody molecules can be produced in vitro or in vivo.

Preferably, the human antibody or antibody fragment comprises an antibody light chain variable region (VL) that comprises three complementarity determining regions or domains and an antibody heavy chain variable region (VH) that comprises three complementarity determining regions or domains. Said VL and VH generally form the antigen binding site. The “complementarity determining regions” (CDRs) are the variable loops of β-strands that are responsible for binding to the antigen. Structures of CDRs have been clustered and classified by Chothia et al. (J Mol Biol 273 (4): 927-948) and North et al., (J Mol Biol 406 (2): 228-256). In the framework of the immune network theory, CDRs are also called idiotypes.

As used herein “fragment” relating to a polypeptide or polynucleotide means a polypeptide or polynucleotide consisting of only a part of the intact polypeptide sequence and structure, or the nucleotide sequence and structure, of the reference gene. The polypeptide fragment can include a C-terminal deletion and/or N-terminal deletion of the native polypeptide, or can be derived from an internal portion of the molecule. Similarly, a polynucleotide fragment can include a 3′ and/or a 5′ deletion of the native polynucleotide, or can be derived from an internal portion of the molecule.

In a further aspect, there is provided a method of improving the anti-cancer effect of a population of cells comprising tumor infiltrating lymphocytes (TILs) comprising depleting innate lymphoid cells (ILCs) from said population.

Depletion can comprise depleting CD56+CD3− cells from the population or alternatively depleting NKp46+ cells from the population

Preferably, the ILCs are depleted prior to TIL expansion or during TIL expansion protocols, but could also include at the time of TIL administration.

If the depletion is performed during TIL expansion, it may be at an initial TIL expansion phase (high does IL-2 in one of the present examples) or a rapid TIL expansion phase (PBMCs and/or “feeder cells”, anti-CD3 and IL.2 in one of the present examples), the latter typically performed shortly before administration to a patient.

In an aspect, there is provided a method of treating cancer in a patient in need thereof, comprising inhibiting the suppressive effect of CD56⁺CD3⁻ innate lymphoid cells (ILCs) on tumor infiltrating lymphocyte (TIL) propagation or expansion.

In an aspect, there is provided a method of treating cancer in a patient in need thereof, comprising administering to the patient a therapeutically effective amount of antibodies against NKG2D, NKp30, NKp46, or combinations thereof. These antibodies may be used alone or in combination with other therapies.

In an aspect, there is provided antibodies against NKG2D, NKp30, NKp46, or combinations thereof for use in the treatment of cancer.

In an aspect, there is provided a use of antibodies against NKG2D, NKp30, NKp46, or combinations thereof in the preparation of a medicament for the treatment of cancer.

In an aspect, there is provided a pharmaceutical composition comprising of antibodies against NKG2D, NKp30, NKp46, or combinations thereof and a pharmaceutically acceptable carrier.

As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

In an aspect, there is provided a method of treating cancer in a patient in need thereof, comprising administering to the patient a therapeutically effective amount of a compound that decreases the suppressive effect of CD56⁺CD3⁻ innate lymphoid cells (ILCs) on tumor infiltrating lymphocyte (TIL) propagation, expansion or function.

In a further aspect, there is provided a method of predicting a patient outcome in a patient having cancer, or patient being treated or having been treated for cancer, preferably time to recurrence or overall survival, comprising measuring the presence of CD56⁺CD3⁻ innate lymphoid cells (ILCs); and predicting a patient outcome, wherein a relatively higher presence of ILCs is associated with a worse patient outcome and a relatively lower presence of ILCs is associated with a better patient outcome. In an embodiment, the presence of ILCs is measured by measuring their gene expression signature or the protein level expression of at least one of CD56^{hi}, CD16⁻, IL-22⁺, CD94⁺, NKG2D⁺, KIR⁺, NKp44⁻, NKp30⁺, and NKp46⁺.

The advantages of the present invention are further illustrated by the following examples. The examples and their particular details set forth herein are presented for illustration only and should not be construed as a limitation on the claims of the present invention.

### EXAMPLES

### Methods and Materials

### Tissue and Blood Specimens

This study was conducted according to the principles expressed in the Declaration of Helsinki. The Research Ethics Board (REB) of the University Health Network (UHN) approved the study. All patients provided written informed consent for the collection of samples. Fresh tissues were obtained from ovarian patients undergoing standard-of-care surgical procedures (UHN REB #10-0335). Tissues were obtained from the UHN Biospecimen Sciences Program. Blood products for TIL growth and assays were obtained from donors with hemochromatosis who were undergoing therapeutic phlebotomy (UHN REB #06-0129). TIL cultures and cell lines used in assays were routinely tested for mycoplasma contamination.

### Phenotypic and Functional Study Restrictions

Functional and phenotypic experiments were performed using tissue from patients with confirmed, highgrade serous cancer and were chemotherapy-naïve at the time of surgery. Tissues were obtained from initial debulking surgeries. Clinicians providing patient outcome data, diagnosis and analysis of IHC sections were blinded to TIL expansion rates and functional/phenotypic studies.

### Media

The complete medium (CM) for initial TIL expansion was comprised of Iscove's modified Dulbecco's medium (IMDM) (Lonza) with 10% human plasma, 25 mM HEPES (Lonza), 100 U/ml penicillin, 100 μg/ml streptomycin (Lonza), 10 μg/ml gentamicin sulfate (Lonza), 2 mM L-glutamine (Lonza), 5.5×10⁻⁵ M 2-mercaptoethanol (Invitrogen), and 6000 IU/ml human recombinant IL-2 (Novartis). For enzyme dissociation medium, the following were added to IMDM: 1 mg/ml collagenase (Sigma), 100 ug/ml DNase I (pulmozyme, Roche), 10 ug/ml gentamicin sulfate, 2 mM L-glutamine, 1.25 μg/ml amphotericin B, 100 U/ml penicillin, and 100 μg/ml streptomycin. XH Media used for suppression assays consisted of X-Vivo 15 (Lonza) plus 5% human plasma, 100 U/ml penicillin, 100 μg/ml streptomycin (Lonza), and 2 mM L-glutamine (Lonza).

### TIL Cultures

Methods for initial TIL expansion are described in Nguyen et al²⁶. In brief, tissues were processed by mincing into ˜1 mm³ pieces and plated in 24-well tissue culture plates, or by enzymatic dissociation before plating at 1×10⁶ cells per well. Cells were cultured in 2 ml CM (containing 6000 IU/ml of human recombinant IL-2) per well in a humidified incubator with 5% CO₂ at 37° C. During culture, half of the medium from each well was replaced with fresh CM three times a week and wells were maintained at a cell concentration of 0.5-2×10⁶ cells/ml. Each independent TIL culture was generally derived from one parental well; during subsequent expansion, all daughter wells derived from the same parental well were combined, mixed, and re-plated. “Fast” expansion rates refer to TIL cultures that yielded >30×10⁶ cells on or before 4 weeks, “slow” refers to TIL cultures which achieved 2-29×10⁶ cells by 4 weeks, and “no” refers to cultures which had cell yields below 2×10⁶ cells at 4 weeks. The TIL culturing was initially performed to assess whether enough cells could be expanded for adoptive T cell therapy clinical trials. Therefore, these criteria are based on the cell numbers needed within a short (maximum 4 week) time frame to seed “rapid expansion protocols” (REPs) in order to generate enough cells for infusion under clinical protocols. For cultures that were harvested before or after 4 weeks, the counts at the time of harvest were used to estimate whether the culture would have been categorized as “fast”, “slow”, or “no” at the 4 week mark. Therefore, some of the cultures in the “slow” category had >30×10⁶ cells at the time of harvest (>4 weeks in culture).

### Further Expansion of TILs After NK Cell Depletion

For all expansion and functional studies, CD56⁺CD3⁻ cells were also CD14⁻ and CD19⁻. TIL cultures with a high proportion of CD56⁺CD3⁻ cells and a low expansion rate were thawed, re-plated at 5×10⁶ cells/well, and rested in CM (containing 6000 IU/ml IL-2) for 7 days. On day 7, TIL were depleted of CD56⁺CD3⁻ cells by flow cytometry-based sorting. Cultures were then subjected to further expansion in CM in 24-well plates as follows: 1×10⁴ CD56⁺CD3⁻ cell-depleted TIL or non-sorted TIL, 1×10⁶ TM-LCL EBV-transformed B lymphoblastoid cells (kind gift from Dr. Cassian Yee, M. D. Anderson) irradiated with 7500 Gy, 5×10⁶ allogeneic PBMCs irradiated with 45 Gy, 30 ng/mL anti-CD3 (OKT3, Miltenyi Biotec) and 600 IU/ml IL-2. Fresh IL-2-containing CM was added every 2-3 days. Cell counts were performed every 2-3 days in parallel with flow cytometric analysis of CD3, CD4, CD8, and CD56 expression. Cell counts were multiplied by the percentage of cells that were CD3⁻CD56⁺ or CD4⁺ T cells (CD3⁺ CD4⁺CD8⁺) or CD8⁺ T cells (CD3⁺ CD4⁻CD8⁺) to calculate expansion yields.

### Suppression Assays

CD56⁺CD3⁻ cells and CD4⁺ and CD8⁺ T cells were purified by flow cytometry-based sorting (BD Aria). For all suppression assays, CD56⁺CD3⁻ cells were also CD14⁻ and CD19⁻. T cells were labeled with Cell Proliferation Dye (eBioscience) and then stimulated at 1×10⁵ cells/well with anti-CD3 and anti-CD28-coated beads (Invitrogen) in the presence or absence of sorted autologous CD56⁺CD3⁻ cells from the slowly expanding TIL cultures (regulatory CD56⁺CD3⁻ cells) at a 1 CD56⁺CD3⁻ cell:4 T cells ratio. After 72 hours, the number of cells present was determined as was the proportions of cells expressing CD3, CD4, CD8, and CD56. Percentage suppression was calculated using the following formula commonly used to calculate suppression by Tregs:

% Suppression=(1−(TIL+CD56⁺CD3⁻ cells/TIL))×100%

Cytokine suppression was determined by analysis of intracellular cytokine staining of cell-sorted CD4⁺ and CD8⁺ T cells (1×10⁵ cells/well) that had been stimulated for 72 hours with anti-CD3 and anti-CD28-coated beads (Invitrogen) in the presence or absence of autologous purified regulatory CD56⁺CD3⁻ cells. Cell Stimulation Cocktail (eBioscience) was used to re-stimulate T cells for 5-6 hours, with brefeldin A (eBioscience) added halfway through the re-stimulation. Following surface staining, cells were fixed using Cytofix/Cytoperm buffer (BD). Intracellular cytokine staining was performed in Cytoperm buffer (BD) with mAbs against TNF-□ (BD) and IFN-□ (BD), IL-9 (eBioscience), IL-17A (eBioscience), and IL-22 (eBioscience). Samples were acquired on a FACSCanto II (BD) and data were analyzed with FlowJo Software.

For suppression assays involving supernatants from regulatory CD56⁺CD3⁻ cells, supernatants from sorted CD56⁺CD3⁻ cells were added every day for duration of the assay and suppression measured as above.

### RNA Preparation and RNA-Sequencing

CD56⁺CD3⁻CD19⁻CD14⁻ cells from slow growing TIL cultures that were confirmed to suppress TILs in functional assays (regulatory CD56⁺CD3⁻ cells), and CD56⁺CD3⁻ CD19⁻CD14⁻ cells from fast-expanding TIL cultures which did not suppress TILs in functional assays (CD56⁺CD3⁻ cells), were sorted by flow cytometry. RNA was isolated using RNeasy Plus Mini kits (Qiagen). RNA preparations were quantified by High Sensitivity RNA qubit assay (Life Technologies/ThermoFisher) and quality by Agilent Bioananlyzer. All samples in this study showed high RNA quality, having RINs between 8.1 and 9.8. 1.5 ng of total RNA per sample was used for library preparation using SMARTer Stranded Total RNA-seq Kit-Pico Input Mammalian (Clontech Laboratories). The paired-end libraries were sequenced on NextSeq 500 (IIlumina) for 75 cycles. RNA-seq performed by the Princess Margaret Genomics Centre (Toronto, Canada)

### RNA-Seq Data Analysis

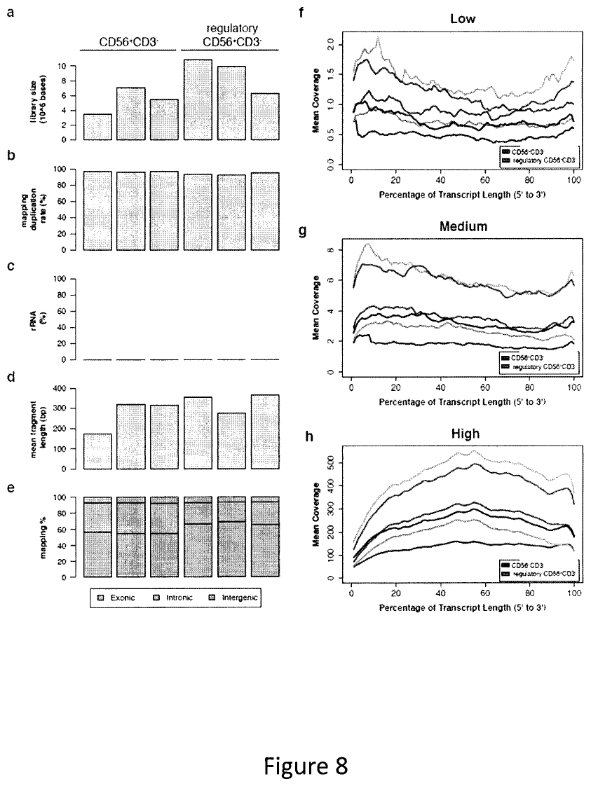

For each sample, raw sequence files in FASTQ format containing an average of 150 million reads were aligned to the GRCh37 human reference genome using STAR v.2.4.2a assisted by the GENCODE v19 transcriptome model annotations⁴². Data alignment quality control measures were collected and verified using RNA-SeQC v1.1.8⁴³. Due to limited DNA input used for sequencing, only highly expressed transcripts could be detected with sufficient sequencing read coverage (See FIG. 8). Gene level transcript abundances were quantified using RSEM v1.2.29 and reported in units of Transcripts Per Million (TPM)⁴⁴. Gene expression heat maps were created using log2-transformed, upper-quartile normalized TPM values using custom scripts in the R statistical environment. DESeq2 R-package was used to perform Principal Component Analysis and identify differentially expressed genes by using expected read counts generated by RSEM ⁴⁵. Differentially expressed genes with p-values adjusted for multiple testing by FDR less than 0.05 and log2 fold-change greater than 1 are reported as statistically significant.

### Flow Cytometric Analyses

Surface marker staining for the following markers was performed in PBS at 4° C. for 30 min following FC block (eBioscience or Biolegend): CD3 (eBioscience, BioLegend or BD) CD4 (eBioscience), CD8, CD56 (BD), CD335 (NKp46) (BioLegend), NKp44 (CD336) (BD), NKp30 (CD337) (BioLegend), CD16 (BD or eBioscience), CD27 (BioLegend), CD158/KIR2DL5 (eBioscience clone #UP-R1), CD57 (eBioscience), CD94 (R&D Systems), NKG2C (CD159c) (R&D Systems, clone #134591), NKG2A (CD159a) (R&D Systems, clone #131411), NKG2D (CD159d) (R&D Systems). KIR3DL1 (BD clone #DX9), KIR2DL3 (R&D Systems, clone #180701), KIR3DL1/3DS1 (Beckman Coulter, clone #Z27), KIR2DL3/2DS2/2DL2 (Merck Research Labs, clone #DX27), KIR3DL2 (Merck Research Labs, clone #DX31), KIR2DS4 (R&D Systems, clone #179315), LIR-1 (HP-F1 generously provided by Dr. Miguel Lopez-Botet) CD19 (BioLegend), CD14 (BioLegend), FOXP3 (clone 236A/E7, eBioscience), and Fixable Viability Dye (eBioscience). Following surface staining, cells were washed and fixed in 2% paraformaldehyde in PBS or BD Cytofix/Cytoperm buffer, depending on the markers analyzed. For all flow-cytometry analysis, CD56⁺CD3⁻ cells were also CD14− and CD19−.

### Cytokine and Chemokine Assays

13-Plex Flow cytomix bead arrays (eBioscience) were used following the manufacturer's instructions to quantify amounts of cytokine in 24-hour supernatants from TIL cultures, which were plated at 1×10⁶/ml in 24-well plates with 6000 IU/ml IL-2 in CM. To quantify secreted cytokines and chemokines, CD56⁺CD3⁻CD19⁻CD14⁻ cells were plated at a concentration of 0.5×10⁶ cells/ml in a 96-well plate in X-Vivo complete media, with and without IL-2 (600 IU/ml). Supernatants were then collected at 24 hours.

### Cytotoxicity Assay

CD56⁺CD3⁻CD19⁻CD14⁻ cells from slow growing TIL cultures that suppressed TILS in functional assays (regulatory CD56⁺CD3⁻ cells), and CD56⁺CD3⁻CD19⁻CD14⁻ cells from peripheral blood of healthy donors (PB NK cell), were isolated by flow cytometry-based sorting and co-cultured with K562 cells (ATCC) in the presence of IL-2. Percent CD107a expression by CD56⁺CD3⁻ cells and fold increase in expression of fixable viability dye (eBioscience) by K562 cells were analyzed after 6 hours.

### Analysis of Publically Available Microarray Data

NCAM1 (CD56) gene expression from the Tothill dataset of 215 HGSC patients⁴⁶ were ranked from high to low, and Kaplan-Meier curves were generated using the corresponding overall survival and recurrence-free survival data (CD56 high n=107, CD56 low n=107). Caveats of using CD56 as a marker, however, include that ˜5% of HGSC tumors we examined by IHC were CD56⁺ and non-CD56⁺ ILCs are not captured with this marker.

### Statistical Analysis

Statistical significance was determined by two-tailed Mann Whitney test or Wilcoxon matched-pairs signed rank test. For Kaplan-Meir curves, significance was determined by Log-rank (Mantel-Cox) test. The n values used to calculate statistics are defined and indicated in figure legends. Significance indicated within figures, and if differences were not significant (p>0.05), this is denoted by n.s.

### Results and Discussion

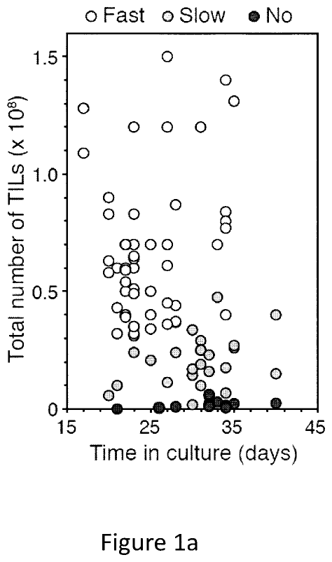

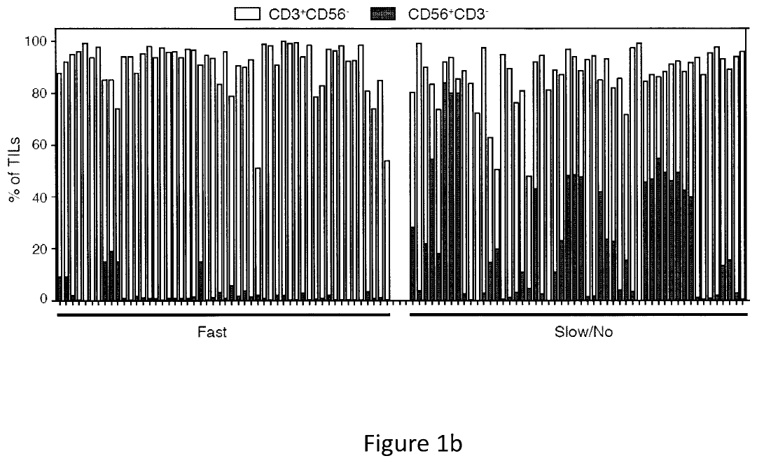

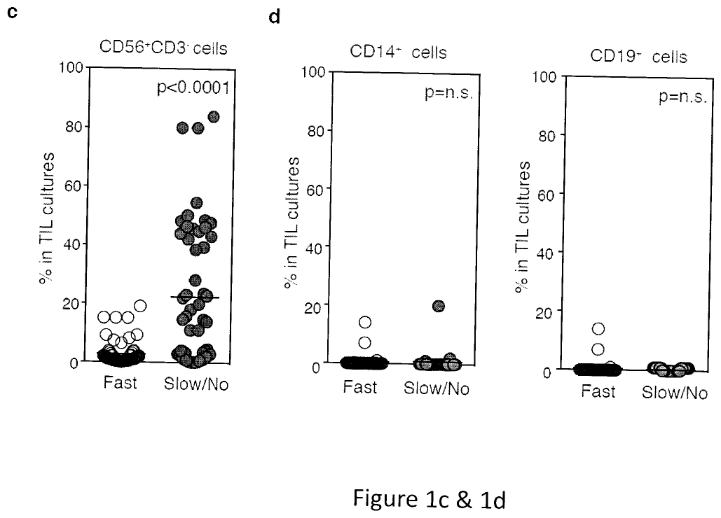

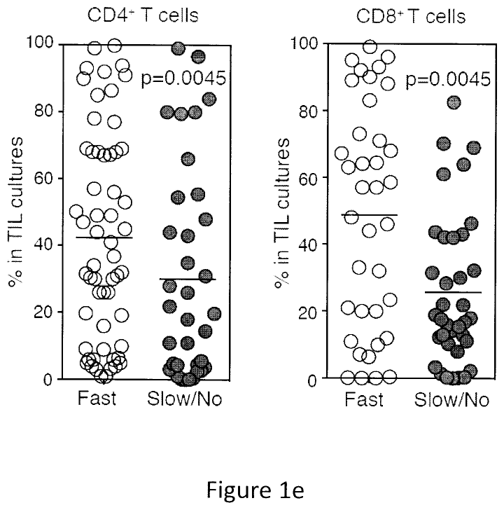

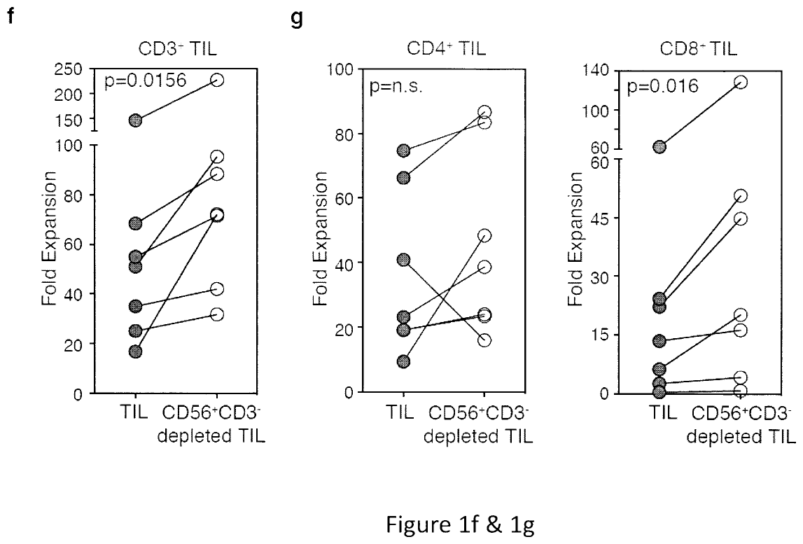

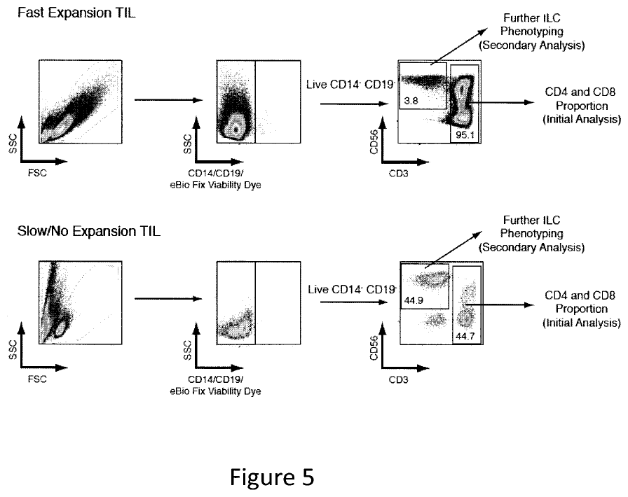

While evaluating the potential of TIL-based adoptive T cell therapy for ovarian cancer, we observed a correlation between the presence of CD56⁺CD3⁻ cells and poor TIL expansion. TIL cultures from primary high-grade serous cancer (HGSC) were grown using established protocols²⁶, and expansion rates and the phenotype of cells present within TIL cultures were assessed (FIG. 1a-e, FIG. 5). A considerable proportion of HGSC TIL cultures grew slowly or failed to expand (FIG. 1a), and would therefore not meet the criteria for use in adoptive cell therapy. TIL cultures that grew slowly generally corresponded to cultures with a high proportion of CD56⁺CD3⁻ cells (FIG. 1b & c), whereas no association with growth rate was observed for CD14⁺ or CD19⁺ populations (FIG. 1d). Further analysis demonstrated that a high proportion of CD56⁺CD3⁻ cells was associated with a reduction in the proportion of CD4⁺ TIL, and to a greater degree, the proportion of CD8⁺ TIL (FIG. 1e). Both fast and slow/no expansion TIL cultures exhibited a range in proportion of CD56⁺CD3⁻ cells, and the proportion of CD56⁺CD3⁻ cells did not have a linear correlation with the expansion rate, suggesting that CD56⁺CD3⁻ cells in “slow/no expansion” TIL cultures differed in their function.

To address the possibility that some patients had suppressive CD56⁺CD3⁻ cells, slow/no expansion TIL cultures were cultured with and without depletion of CD56⁺CD3⁻ cells, together with irradiated feeder cells, anti-CD3 mAb, and IL-2. This is similar to protocols used to rapidly expand TIL cultures immediately prior to cell infusion in clinical trials. TIL expansion increased in the absence of the CD56⁺CD3⁻ cells (FIG. 1f). In the majority of patients, an increase in CD4⁺ and CD8⁺ TIL expansion was observed in the absence of the CD56⁺CD3⁻ cells but a statistically significant expansion rate was noted only for CD8⁺ TILs (FIG. 1g).

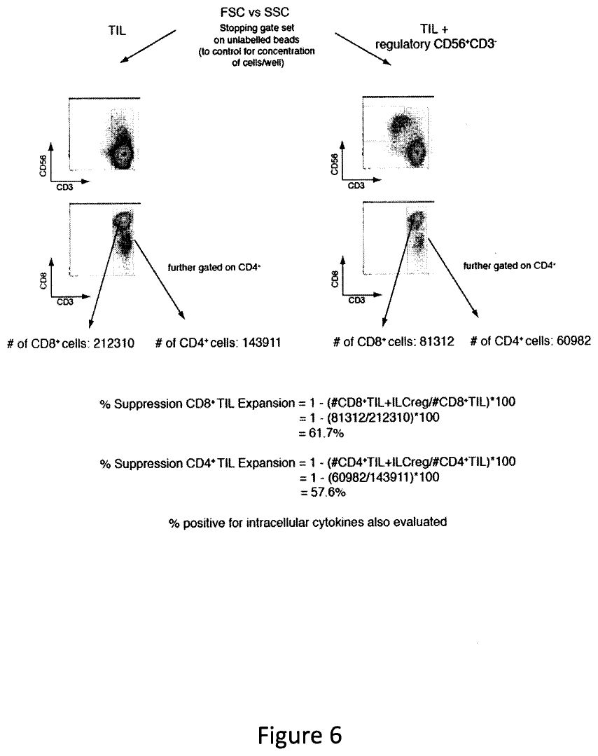

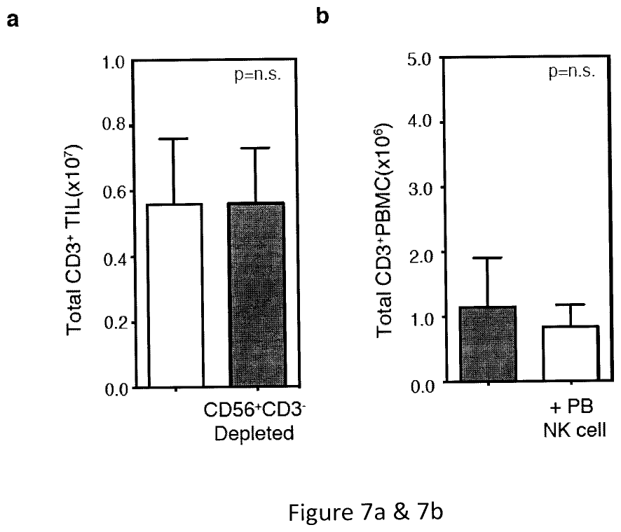

To examine whether these CD56⁺CD3⁻ cells could suppress T cells that received a ‘strong’ proliferative signal, and evaluate whether suppression was linked to the presence of antigen-presenting cells (APCs) or IL-2, we performed assays similar to in vitro regulatory T cell (Treg) suppression assays. TIL cultures that did not expand well were depleted of CD56⁺CD3⁻ cells and then activated with anti-CD3 and anti-CD28-coated beads. CD56⁺CD3⁻ cells were then added back at a ratio of one CD56⁺CD3⁻ cell to four T cells. The addition of CD56⁺CD3⁻ cells suppressed CD4⁺ and CD8⁺ TIL expansion in the absence of APCs or exogenous IL-2 (FIG. 1h and FIG. 6), indicating that CD56⁺CD3⁻ cells from cultures with impaired TIL expansion were capable of directly suppressing T cell proliferation. This capacity to limit T cell expansion was not shared by CD56⁺CD3⁻ cells from fast-expanding TIL cultures or when peripheral blood (PB) NK cells were co-cultured with autologous PB T cells (FIG. 7a-b), supporting the possibility that CD56⁺CD3⁻ cells from slowly expanding TIL cultures were a distinct regulatory population.

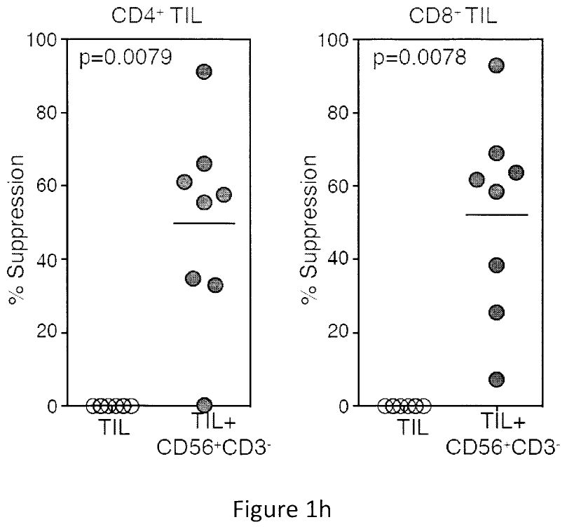

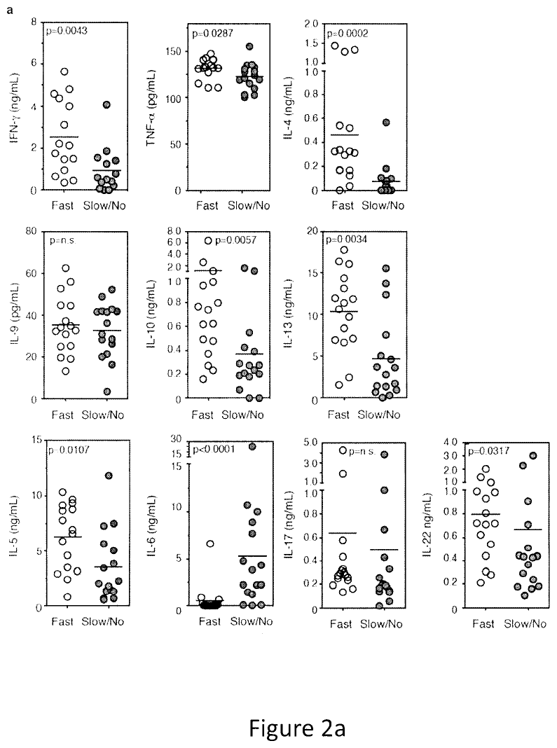

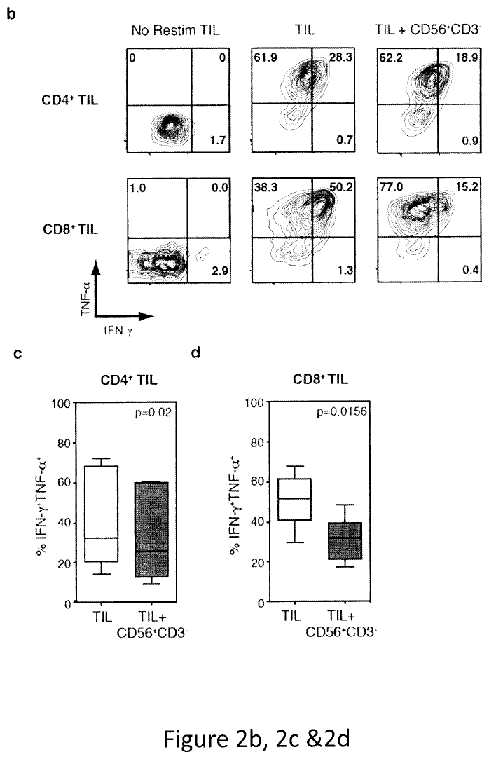

NK cells and other ILCs can contribute to the initiation and polarization of the adaptive immune response^{4, 5}, therefore experiments were done to evaluate cytokine production in slow/no versus rapidly expanding TIL cultures. TIL cultures that exhibited slow/no expansion and also contained a high proportion of CD56⁺CD3⁻ cells had lower amounts of IFN-□, TNF-□, IL-4, IL-5, IL-10, and IL-13, but higher amounts of IL-6 (FIG. 2a). To determine if CD56⁺CD3⁻ cells from slow/no expansion TIL cultures directly regulated TIL cytokine production, sorted CD56⁺CD3⁻ cells were co-cultured with autologous CD3⁺CD56⁻ TIL and activated with anti-CD3 and anti-CD28 coated beads. The percentages of CD4⁺ and CDS⁺ TIL that were TNF-□⁺IFN-□⁺ were lower in the presence of CD56⁺CD3⁻ cells (FIG. 2b-d), with a clear reduction in IFN-□ production.

Thus, CD56⁺CD3⁻ cells from slow/no expansion TIL cultures also modulated cytokines produced by CD4⁺ and CD8⁺ TIL.

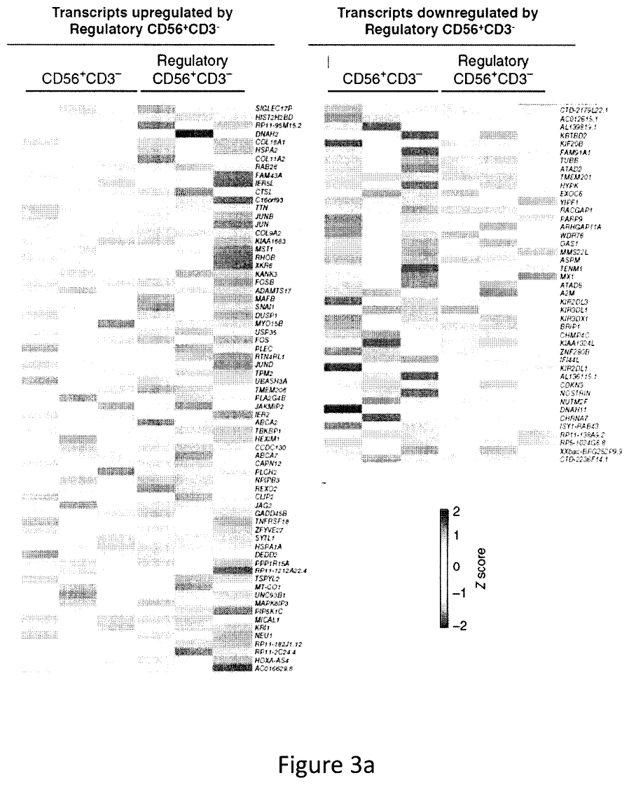

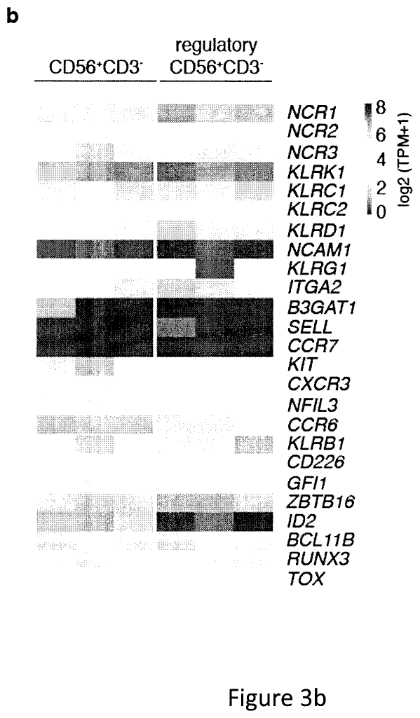

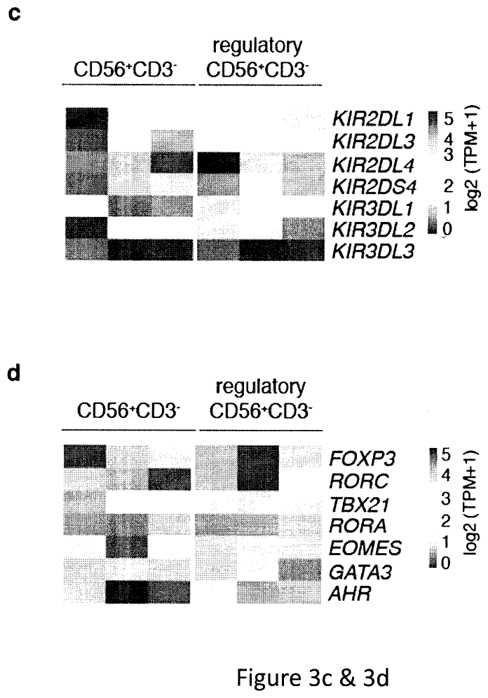

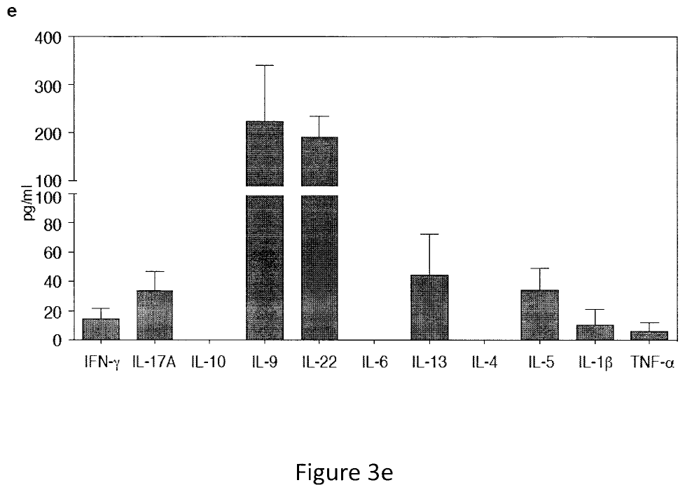

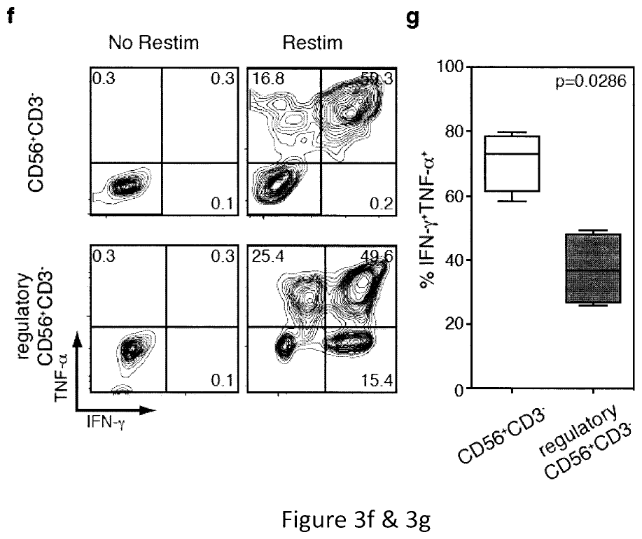

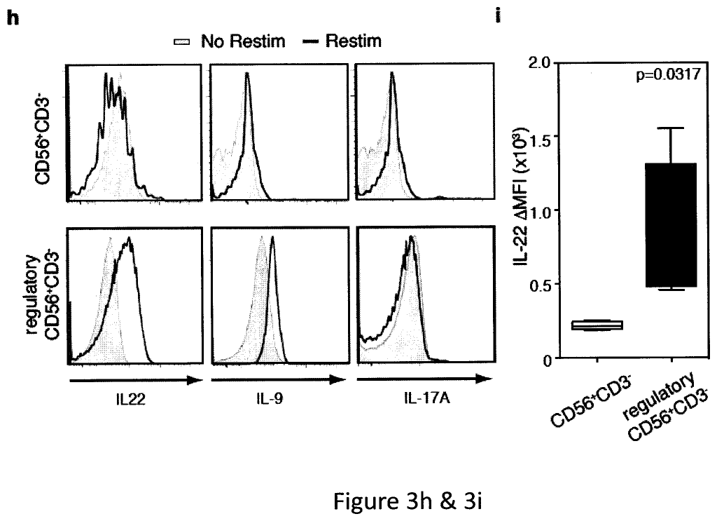

To interrogate unique and overlapping properties between suppressive CD56⁺CD3⁻ cells from slow/no expansion TIL cultures (regulatory CD56⁺CD3⁻) and non-suppressive CD56⁺CD3⁻ cells from fast-expanding TIL cultures (CD56⁺CD3⁻), we performed transcriptome profiling of these populations from 6 independent donors using RNA-seq (FIG. 3a-d, FIG. 8). Comparison of gene-level expression fold-change between the two CD56⁺CD3⁻ cell populations revealed a set of statistically significant differentially expressed genes that distinguished regulatory CD56⁺CD3⁻ cells from non-regulatory CD56⁺CD3⁻ cells, confirming that these two populations were distinct from one another (FIG. 3a). Transcriptome profiles of regulatory CD56⁺CD3⁻ cells from three independent donors were remarkably similar, supporting a unique but shared pattern of gene expression amongst different individuals. When expression of NK cell and ILC-associated molecules were examined, both populations had high transcript level expression of NK cell-associated genes, including natural cytotoxicity receptors (NCR), NKG2-CD94 family, and killer-cell immunoglobulin-like receptors (KIRs) (FIG. 3b-c). High expression of NCR1 (NKp46), NCR3 (NKp30), KLRK1 (NKG2D), KLRC1 (NKG2A), KLRD1 (CD94), KIRs and CD7, and low CD16 expression, was confirmed by flow cytometry (FIG. 9, 10). Regulatory CD56⁺CD3⁻ cells also had high expression of ID2, ZBTB16 (PLZF), KLRB1 (CD161), RUNX3, TOX and KIT (CD117), and low or no detectible expression of SELL (CD62L), B3GAT1 (CD57), ITGA2 (CD49b). Interestingly, regulatory CD56⁺CD3⁻ and non-regulatory CD56⁺CD3⁻ populations exhibited high transcript level expression of EOMES, TBX21, GATA3, RORA and AHR, a transcription factor expression profile which overlaps with NK cells, ILC2s, and ILC3s²⁷ (FIG. 3d). While the regulatory CD56⁺CD3⁻ population was able to suppress anti-tumor T cells, FOXP3 could not be detected at either transcript or protein level (FIG. 3d, FIG. 11), indicating this Treg lineage-defining transcription factor^{28, 29, 30} is not required for ILC-mediated suppression.

To examine cytokine production by the regulatory CD56⁺CD3⁻ population, CD56⁺CD3⁻ cells were sorted from slow/no expansion TIL cultures and cultured overnight in IL-2. Regulatory CD56⁺CD3⁻ cells produced minimal interferon (IFN)-γ, but secreted high amounts of IL-9 and IL-22, and low amounts of IL-5, IL-13, and IL-17A (FIG. 3e). Despite producing cytokines characteristic of ILC2 and ILC3, regulatory CD56⁺CD3⁻ cells produced very high levels of CCL3 (FIG. 12), reportedly expressed by ILC1s but not by ILC3s³¹.

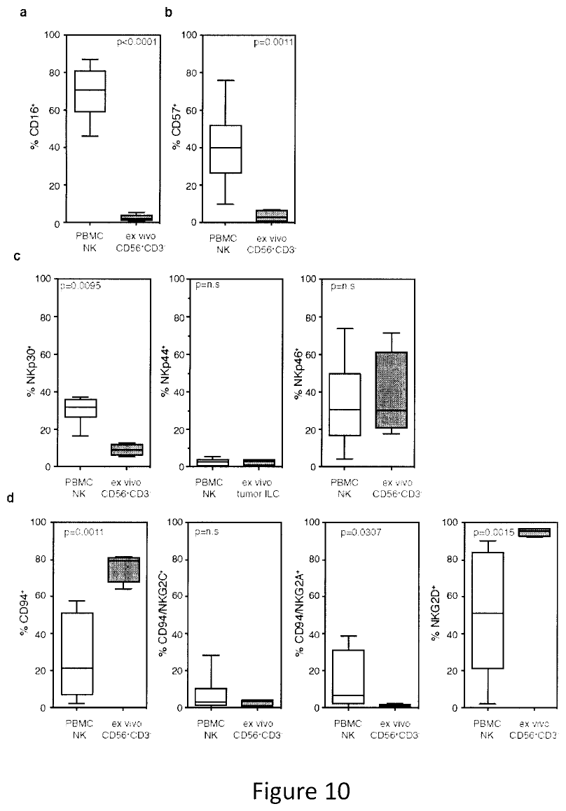

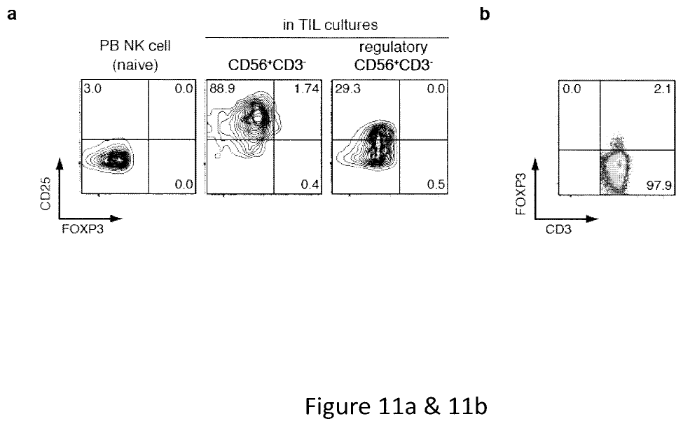

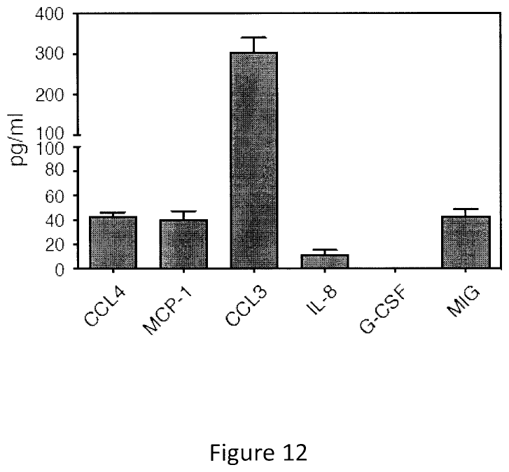

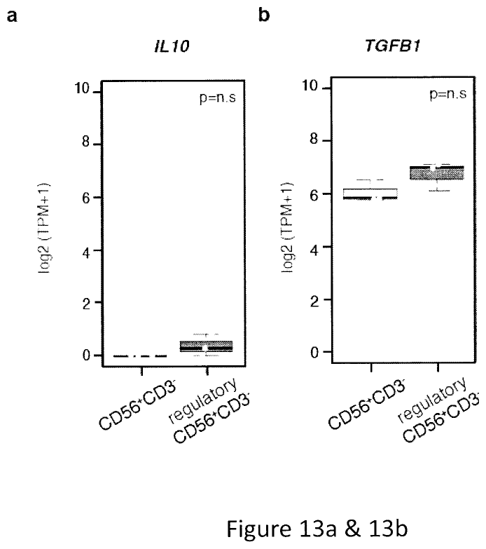

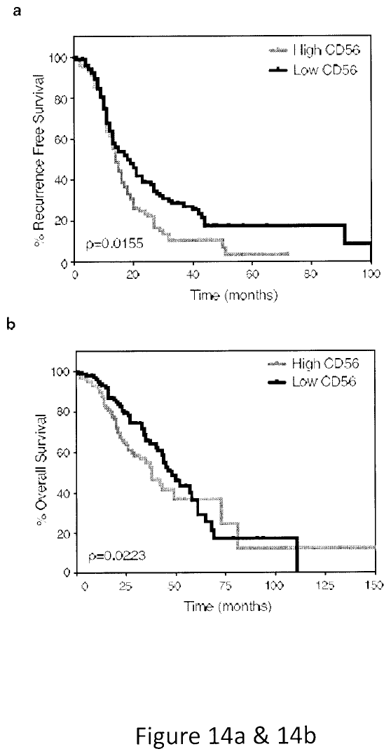

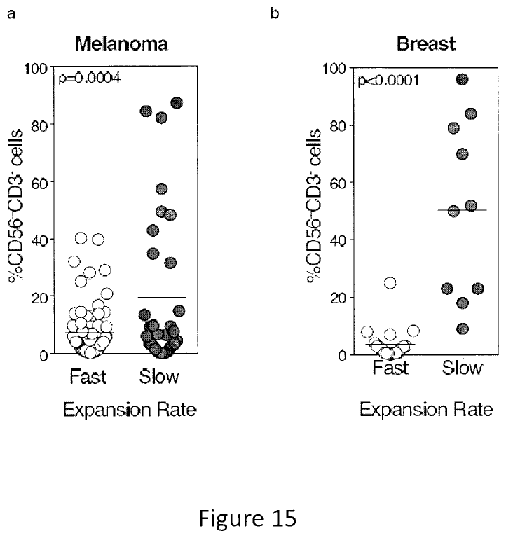

Cytokine expression was further assessed by intracellular cytokine staining. Sorted CD56⁺CD3⁻ cells were re-stimulated with PMA and ionomycin for 5-6 hours. TNF-α and IFN-γ expression could be induced in the ILC populations, however, the two ILC populations differed in the proportions of cells that expressed either TNF-α alone, IFN-γ alone, or co-expressed both TNF-α and IFN-γ (FIG. 3f-g). Stimulated regulatory CD56⁺CD3⁻ cells produced IL-22, whereas non-regulatory CD56⁺CD3⁻ cells did not, supporting that expression of this cytokine may be a characteristic of regulatory CD56⁺CD3⁻ cells (FIG. 3h-i). Yet ex vivo, regulatory CD56⁺CD3⁻ cells were NKp44⁻ (FIG. 10c), distinguishing them from ILC3s⁷. Low IL-9 expression was detected by regulatory CD56⁺CD3⁻ cells in three of five donors, but neither regulatory CD56⁺CD3⁻ nor non-regulatory CD56⁺CD3⁻ cells produced detectable IL-17A under these conditions (FIG. 3h). Therefore, this regulatory CD56⁺CD3⁻ population expresses many NK cell-associated receptors, but clearly has unique features, including a gene expression signature that distinguishes these cells from non-suppressive CD56⁺CD3⁻ cells, and a unique cytokine and chemokine profile than that of other described ILCs.

A variety of mechanisms have been reported that govern NK cell-mediated T cell regulation. An IL-2- and contact-dependent mechanism was reported with NK cell regulation of T cell responses to human parainfluenza virus type 3 infection³¹. Other studies have observed IL-10-mediated suppression, indirect suppression by impacting DCs, and suppression via receptors including 2B4, NKG2D and NKp46^{17, 19, 20, 21}. RNAseq analysis showed that there were high levels of transcripts associated with cytotoxicity, including granzyme A, granzyme B, and perforin in the regulatory CD56⁺CD3⁻ population (FIG. 4a). We, therefore, assessed the cytotoxic potential of these cells. CD56⁺CD3⁻ cells were sorted from TIL cultures that had expanded slowly and were co-cultured with K562 target cells in the presence of IL-2. Regulatory CD56⁺CD3⁻ cells had low CD107a expression after co-culturing and induced minimal K562 cell death (FIG. 4b-d). In comparison, PB NK cells expressed high levels of CD107a and mediated significant cytotoxicity against K562 cells.

IL-10 expression by regulatory CD56⁺CD3⁻ cells was not observed at either the transcript or protein level (FIG. 3e, FIG. 13a), and there was no increase in TGFB1 transcript levels compared to non-regulatory CD56⁺CD3⁻ cells (FIG. 13b). The unique cytokine expression profile of regulatory CD56⁺CD3⁻ cells, however, suggested that regulatory CD56⁺CD3⁻ cells might suppress T cells via a secreted factor. To address this possibility, CD56⁺CD3⁻ cells from slow/no expansion TIL cultures were sorted by flow cytometry, plated, and supernatants were collected at 16 hours. Expansion and cytokine production of autologous flow cytometry-sorted CD3⁺CD56⁻ TIL was then assessed after incubation with and without the supernatants from regulatory CD56⁺CD3⁻ cells. No difference in either the proliferation or cytokine production was observed (FIG. 4e-f).

Regulatory CD56⁺CD3⁻ cells had high transcript and protein level expression of NKG2D (KLRK1), as well as NKp30 (NCR3) and NKp46 (NCR1) (FIG. 4g-h), two NCRs implicated in the interaction between NK cells and other immune cells^{21, 32, 33}. We therefore, examined whether these receptors were involved in TIL suppression. TIL cultures with high proportions of CD56⁺CD3⁻ cells and slow/no expansion were activated with irradiated feeder cells, anti-CD3 mAb, and IL-2 in the presence or absence of anti-NKG2D, anti-NKp30, and anti-NKp46 antibodies (FIG. 4i). Addition of anti-NKG2D to the cultures increased T cell expansion in six of seven patients. As T cells also express NKG2D, and anti-NKG2D can be an agonist for T cells, the effects of anti-NKG2D on T cells versus regulatory CD56⁺CD3⁻ cells could not be distinguished. However, neither NKp30 nor NKp46 are expressed by T cells. Addition of anti-NKp30 increased expansion yields of T cells in four of seven patients, but reduced expansion in two patients. Importantly, anti-NKp46 treatment resulted in comparable T cell expansion yields to those achieved by depletion of CD56⁺CD3⁻ cells in all seven patients (FIG. 1f), demonstrating that anti-NKp46 interferes with the activity of regulatory CD56⁺CD3⁻ cells. Therefore, NKG2D, NKp30, and particularly NKp46 interactions may promote the suppression of TIL expansion that is mediated by regulatory CD56⁺CD3⁻ cells.

The ability of regulatory CD56⁺CD3⁻ cells to suppress autologous TIL suggested these patients might have reduced immune surveillance. To examine this possibility, we evaluated whether the presence of regulatory CD56⁺CD3⁻ cells in TIL cultures corresponded to a difference in clinical outcomes for HGSC patients compared to patients with fast TIL expansion that did not have a population of regulatory CD56⁺CD3⁻ cells. When recurrence-free survival (RFS) was examined, the average time to recurrence was 12.6 months for patients with regulatory CD56⁺CD3⁻ cells in their TIL cultures versus 24 months for patients who did not have regulatory CD56⁺CD3⁻ cells in their TIL cultures (FIG. 4j). The presence of regulatory ILCs in TIL cultures therefore corresponded to a shorter time to relapse. While we did not repeat these studies in an independent cohort, high CD56 expression in the annotated microarray data set published by Tothill et al³⁴, was associated with a significant reduction in RFS (FIG. 14), supporting the interpretation that CD56⁺CD3⁻ cells may be a negative prognostic biomarker for HGSC.

Our findings that some HGSC patients have TIL cultures containing ILCregs but other patients do not, suggests that the tumor microenvironment may play a role in recruiting and or promoting the differentiation of immunosuppressive CD56⁺CD3⁻ cells. It is important to note that this association is not restricted to HGSC, as we have observed that a high proportion of CD56⁺CD3⁻ cells in melanoma and breast TIL cultures is also associated with poor TIL expansion (FIG. 15). In the context of TIL-based adoptive cell therapy, depletion of CD56⁺CD3⁻ cells in expansion protocols may represent a novel method to improve this immunotherapy.

The regulatory CD56⁺CD3⁻ cells that we describe are CD56^{hi} CD16⁻, CD94⁺, NKG2D⁺, KIR⁺, NKp44⁻ ex vivo, NKp30⁺, NKp46⁺ lymphocytes that can produce IL-22 when stimulated ex vivo, and that limit T cell cytokine production and expansion. While capable of making IFN-γ and TNF-α, regulatory CD56⁺CD3⁻ cells are not actively secreting these cytokines in IL-2-expanded TIL cultures. The majority of cultures contained a high proportion of CD94⁺ cells and expressed various KIRs, which would point to these cells being of NK cell origin. However, other cultures displayed differences in expression of NK cell-associated molecules, leaving the possibility of a heterogeneous CD56⁺CD3⁻ ILC population in these individuals. However, our study clearly demonstrates that the ability of regulatory CD56⁺CD3⁻ cells to suppress TILs involves NKp46, supporting a role for this NCR in regulating interactions with T cells.

Importantly, ILCs and NK cells with immunosuppressive capacity in our study and others have been found to share many of the same characteristics of Tregs. In addition to suppressing T cell expansion and cytokine production, some models have shown that suppressive NK cells/ILCs produce IL-10^{35, 36}, inhibit B cell function and memory^{37, 38}, dampen immune responses by modulating dendritic cell function³⁶⁻⁴⁰, as well as limit immunity by killing CD8⁺ T cells^{19, 20}. While the majority of studies have described immunosuppressive ILCs as NK cells, various shared and distinct properties of suppressive ILCs compared to conventional NK cells and other ILC subsets is not well defined. From this perspective, the origin and differentiation of regulatory ILCs must be better understood. Importantly for human disease, the extent to which ILCs regulate immune responses in a multitude of contexts should be evaluated.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1 Smyth, M. J., Ngiow, S. F., Ribas, A. & Teng, M. W. Combination cancer immunotherapies tailored to the tumour microenvironment. Nature reviews. Clinical oncology 13, 143-158, (2016).

2 Callahan, M. K., Postow, M. A. & Wolchok, J. D. Targeting T Cell Co-receptors for Cancer Therapy. Immunity 44, 1069-1078, (2016).

3 Schildberg, F. A., Klein, S. R., Freeman, G. J. & Sharpe, A. H. Coinhibitory Pathways in the B7-CD28 Ligand-Receptor Family. Immunity 44, 955-972, (2016).

4 Crome, S. Q., Lang, P. A., Lang, K. S. & Ohashi, P. S. Natural killer cells regulate diverse T cell responses. Trends Immunol 34, 342-349, (2013).

5 Gasteiger, G. & Rudensky, A. Y. Interactions between innate and adaptive lymphocytes. Nat Rev Immunol 14, 631-639, (2014).

6 Artis, D. & Spits, H. The biology of innate lymphoid cells. Nature 517, 293-301, (2015).

7 Spits, H. et al. Innate lymphoid cells—a proposal for uniform nomenclature. Nat Rev Immunol 13, 145-149, (2013).

8 Spits, H., Bernink, J. & Lanier, L. L. NK cells and type 1 innate lymphoid cells: partners in host defense. Nat Immunol 17, 758-764 (2016).

9 Takeda, K. & Dennert, G. The development of autoimmunity in C57BL/6 Ipr mice correlates with the disappearance of natural killer type 1-positive cells: evidence for their suppressive action on bone marrow stem cell proliferation, B cell immunoglobulin secretion, and autoimmune symptoms. J Exp Med 177, 155-164 (1993).

10 Villanueva, J. et al. Natural killer cell dysfunction is a distinguishing feature of systemic onset juvenile rheumatoid arthritis and macrophage activation syndrome. Arthritis research & therapy 7, R30-37, (2005).

11 Zhou, R. B., Wei, H. M. & Tian, Z. G. NK3-like NK cells are involved in protective effect of polyinosinic-polycytidylic acid on type I diabetes in nonobese diabetic mice. J Immunol 178, 2141-2147 (2007).

12 Tai, L. H. et al. Positive regulation of plasmacytoid dendritic cell function via Ly49Q recognition of class I MHC. J Exp Med 205, 3187-3199, (2008).

13 Beilke, J. N., Kuhl, N. R., Van Kaer, L. & Gill, R. G. NK cells promote islet allograft tolerance via a perforin-dependent mechanism. Nat Med 11, 1059-1065, (2005).

14 Noval Rivas, M. et al. NK cell regulation of CD4 T cell-mediated graft-versus-host disease. J Immunol 184, 6790-6798, (2010).

15 Su, H. C. et al. NK cell functions restrain T cell responses during viral infections. Eur J Immunol 31, 3048-3055, (2001).

16 Noone, C. M. et al. Natural killer cells regulate T-cell proliferation during human parainfluenza virus type 3 infection. J Virol 82, 9299-9302, (2008).

17 Waggoner, S. N., Taniguchi, R. T., Mathew, P. A., Kumar, V. & Welsh, R. M. Absence of mouse 2B4 promotes NK cell-mediated killing of activated CD8+ T cells, leading to prolonged viral persistence and altered pathogenesis. J Clin Invest 120, 1925-1938, (2010).

18 Soderquest, K. et al. Cutting edge: CD8+ T cell priming in the absence of NK cells leads to enhanced memory responses. J Immunol 186, 3304-3308, (2011).

19 Waggoner, S. N., Cornberg, M., Selin, L. K. & Welsh, R. M. Natural killer cells act as rheostats modulating antiviral T cells. Nature 481, 394-398, (2012).

20 Lang, P. A. et al. Natural killer cell activation enhances immune pathology and promotes chronic infection by limiting CD8+ T-cell immunity. Proc Natl Acad Sci USA 109, 1210-1215, (2012).

21 Narni-Mancinelli, E. et al. Tuning of natural killer cell reactivity by NKp46 and Helios calibrates T cell responses. Science 335, 344-348, (2012).

22 Peppa, D. et al. Up-regulation of a death receptor renders antiviral T cells susceptible to NK cell-mediated deletion. J Exp Med 210, 99-114, (2013).

23 Ahlenstiel, G. et al. Early changes in natural killer cell function indicate virologic response to interferon therapy for hepatitis C. Gastroenterology 141, 1231-1239, 1239 e1231-1232, (2011).

24 Munneke, J. M. et a/. Activated innate lymphoid cells are associated with a reduced susceptibility to graft-versus-host disease. Blood 124, 812-821, (2014).

25 Hepworth, M. R. et a/. Innate lymphoid cells regulate CD4+ T-cell responses to intestinal commensal bacteria. Nature 498, 113-117, (2013).

26 Nguyen, L. T. et al. Expansion and characterization of human melanoma tumor-infiltrating lymphocytes (TILs). PLoS One 5, e13940, (2010).

27 Koues, O. I. et al. Distinct Gene Regulatory Pathways for Human Innate versus Adaptive Lymphoid Cells. Cell 165, 1134-1146, (2016).

28 Hori, S., Nomura, T. & Sakaguchi, S. Control of regulatory T cell development by the transcription factor Foxp3. Science 299, 1057-1061, (2003).

29 Fontenot, J. D., Gavin, M. A. & Rudensky, A. Y. Foxp3 programs the development and function of CD4+CD25+ regulatory T cells. Nat Immunol 4, 330-336, (2003).

30 Khattri, R., Cox, T., Yasayko, S. A. & Ramsdell, F. An essential role for Scurfin in CD4⁺CD25⁺ T regulatory cells. Nat Immunol 4, 337-342 (2003).

31 Bernink, J. H. et al. Human type 1 innate lymphoid cells accumulate in inflamed mucosal tissues. Nat Immunol 14, 221-229, (2013).

32 Roy, S. et al. NK cells lyse T regulatory cells that expand in response to an intracellular pathogen. J Immunol 180, 1729-1736 (2008).

33 Simhadri, V. R. et al. Dendritic cells release HLA-B-associated transcript-3 positive exosomes to regulate natural killer function. PLoS One 3, e3377, (2008).

34 Tothill, R. W. et al. Novel molecular subtypes of serous and endometrioid ovarian cancer linked to clinical outcome. Clinical cancer res 14, 5198-5208, (2008).

35 Lee, S. H., Kim, K. S., Fodil-Cornu, N., Vidal, S. M. & Biron, C. A. Activating receptors promote NK cell expansion for maintenance, IL-10 production, and CD8 T cell regulation during viral infection. J Exp Med 206, 2235-2251, (2009).

36 Fodil-Cornu, N. et al. Ly49h-deficient C57BL/6 mice: a new mouse cytomegalovirus-susceptible model remains resistant to unrelated pathogens controlled by the NK gene complex. J Immunol 181, 6394-6405 (2008).

37 Rydyznski, C. E. & Waggoner, S. N. Boosting vaccine efficacy the natural (killer) way. Trends Immunol 36, 536-546, (2015).

38 Che, S. & Huston, D. P. Natural killer cell suppression of IgM production. Natural immunity 13, 258-269 (1994).

39 Della Chiesa, M. et al. The natural killer cell-mediated killing of autologous dendritic cells is confined to a cell subset expressing CD94/NKG2A, but lacking inhibitory killer Ig-like receptors. Eur J Immunol 33, (2003).

40 Piccioli, D., Sbrana, S., Melandri, E. & Valiante, N. M. Contact-dependent stimulation and inhibition of dendritic cells by natural killer cells. J Exp Med 195, 335-341 (2002).

1. A method of improving the anti-cancer effect of a population of cells comprising tumor infiltrating lymphocytes (TILs), the method comprising adding to said population a compound that decreases the suppressive effect of CD56⁺CD3⁻ innate lymphoid cells (ILCs) on tumor infiltrating lymphocyte (TIL) propagation, expansion or function.

2. The method of claim 1, wherein the cancer is a cancer associated with poor TIL expansion.

3. The method of claim 1, wherein the cancer is melanoma, breast cancer, prostate cancer, or ovarian cancer.

4. (canceled)

5. The method of claim 1, wherein the ILCs are at least one of CD56^{hi}, CD16⁻, CD94⁺, NKG2D⁺, KIR⁺, NKp44⁻ ex vivo, NKp30⁺, NKp46⁺.

6. The method of claim 1, wherein the ILCs are all of CD56^{hi}, CD16⁻, CD94⁺, NKG2D⁺, NKp44⁻ ex vivo, NKp30⁺, NKp46⁺.

7. The method of claim 1, wherein the compound is an antibody against a surface marker on CD56⁺CD3⁻ ILCs.

8.-14. (canceled)

15. A method of treating cancer in a patient in need thereof, comprising inhibiting the suppressive effect of CD56⁺CD3⁻ innate lymphoid cells (ILCs) on tumor infiltrating lymphocyte (TIL) propagation or expansion.

16. A method of treating cancer in a patient in need thereof, comprising administering to the patient a therapeutically effective amount of antibodies against NKG2D, NKp30, NKp46, or combinations thereof.

17. The method of claim 16, wherein the antibodies are used alone.

18. The method of claim 16, wherein the antibodies are used in combination with other therapies.

19.-20. (canceled)

21. A pharmaceutical composition comprising of antibodies against NKG2D, NKp30, NKp46, or combinations thereof, and a pharmaceutically acceptable carrier.

22. A method of treating cancer in a patient in need thereof, comprising administering to the patient a therapeutically effective amount of a compound that decreases the suppressive effect of CD56⁺CD3⁻ innate lymphoid cells (ILCs) on tumor infiltrating lymphocyte (TIL) propagation, expansion or function.

23. The method of claim 22, wherein the cancer is a cancer associated with poor TIL expansion.

24. The method of claim 22, wherein the cancer is melanoma, breast cancer, prostate cancer, or ovarian cancer.

25. The method of claim 24, wherein the ovarian cancer is serous ovarian cancer.

26. The method of claim 22, wherein the ILCs are at least one of CD56^{hi}, CD16⁻, CD94⁺, NKG2D⁺, KIR⁺, NKp44⁻ ex vivo, NKp30⁺, NKp46⁺.

27. The method of claim 22, wherein the ILCs are all of CD56^{hi}, CD16⁻, CD94⁺, NKG2D⁺, KIR⁺, NKp44⁻ ex vivo, NKp30⁺, NKp46⁺.

28. The method of claim 22, wherein the compound is an antibody against a surface marker on CD56⁺CD3⁻ ILCs.

29.-30. (canceled)

31. The method of claim 7, wherein the compound is NKG2D, NKp30, NKp46, or combinations thereof.

32. The method of claim 28, wherein the compound is NKG2D, NKp30, NKp46, or combinations thereof.
